SEQUENCE LISTING (18 sequences)

SEQ ID NO: 1 - DNA, 21 bp, Artificial Sequence
  primer
gcgaacttaccaagtctctgc

SEQ ID NO: 2 - DNA, 21 bp, Artificial Sequence
  primer
ggtccaggattctgcctatga

SEQ ID NO: 3 - DNA, 21 bp, Artificial Sequence
  primer
tggacgagctgggcaaagtgc

SEQ ID NO: 4 - DNA, 21 bp, Artificial Sequence
  primer
cctgctcgtacaccagccaga

SEQ ID NO: 5 - DNA, 21 bp, Artificial Sequence
  primer
ctcagaacttgatccctgccc

SEQ ID NO: 6 - DNA, 21 bp, Artificial Sequence
  primer
ccagccctggagcttggaaca

SEQ ID NO: 7 - DNA, 21 bp, Artificial Sequence
  primer
tgcagagtgtagtgcctcacc

SEQ ID NO: 8 - DNA, 21 bp, Artificial Sequence
  primer
gcaggttgtccggttcatgat

SEQ ID NO: 9 - DNA, 15 bp, Artificial Sequence
  primer
tgttgtcactctcct

SEQ ID NO: 10 - DNA, 21 bp, Artificial Sequence
  primer
ccagcgactgtgttcacccat

SEQ ID NO: 11 - DNA, 21 bp, Artificial Sequence
  primer
tatcgctcagcgttcagtgtg

SEQ ID NO: 12 - DNA, 21 bp, Artificial Sequence
  primer
ggcctggtccacattcttttc

SEQ ID NO: 13 - DNA, 23 bp, Artificial Sequence
  primer
acccttcaccaatgactcctatg

SEQ ID NO: 14 - DNA, 21 bp, Artificial Sequence
  primer
atgatgactgcagcaaatcgc

SEQ ID NO: 15 - protein, 17 aa, Mus musculus
KYTSHLDPNTLLDYYLA

SEQ ID NO: 16 - protein, 10 aa, Mus musculus
RVYVGYEAVA

SEQ ID NO: 17 - protein, 13 aa, Mus musculus
KQVANGALGVVYW

SEQ ID NO: 18 - protein, 13 aa, Mus musculus
KGLEDVTVGAYDV

(19) United States
(12) Patent Application Publication
(10) Pub. No.: US 2004/0002112 A1
(43) Pub. Date: Jan. 1, 2004

(54) Proteins involved in regulation of adipocytes and uses related thereto

(76) Inventors: Matthias Mann, Odense (DK); Alexandre V. Podtelejnikov, Odense (DK); Blagoy Andonov Blagoev, Odense (DK); Irina Hristova Kratchmarova, Odense (DK); Akhilesh Pandey, Baltimore, MD (US)

Correspondence Address: ROPES & GRAY LLP, ONE INTERNATIONAL PLACE, BOSTON, MA, 02110-2624, US

(21) Appl. No.: 10/285,335

(22) Filed: Oct. 31, 2002

Related U.S. Application Data
(60) Provisional application No. 60/336,386, filed on Oct. 31, 2001.

(51) Int. Cl. G01N 33/53

(52) U.S. Cl. 435/7.1

(57) ABSTRACT

The invention relates to the identification and quantification of proteins differentially expressed during adipogenesis. More specifically, the invention relates to methods for identifying and/or quantitating proteins differentially expressed in one or more specific stage(s) of adipocyte differentiation, using mass spectrometry. The invention also provides those differentially expressed genes as marker genes of adipogenesis. The invention further provides treatment methods for patients suffering from conditions associated with hyper- or hypo-adipogenesis.

### REFERENCE TO RELATED APPLICATIONS

This application claims priority to U.S. Provisional Application 60/336,386, filed on Oct. 31, 2001, the entire content of which is incorporated herein by reference.

### BACKGROUND OF THE INVENTION

Obesity is a major health concern today. Not only does this condition pose a wide range of psychological affects related to societal perceptions of personal appearance, but it is also a major contributing factor to type II diabetes mellitus, heart disease, hypertension, certain forms of cancer, sleep disorders, respiratory disorders, osteoarthritis, as well as a range of other conditions (Kopelman, 2000; Friedman, 2000). These serious medical maladies result not only in human suffering, but also in the high costs related to lost work and increased medical expenses. Costs of obesity and obesity related illnesses are estimated at approximately 2-7% of total health care costs (Seidell, 1996). Accompanying such morbitity is an increase in mortality resulting from obesity. Data from the Framingham Heart Study estimates that one's risk of death increases by 1% for each extra pound increase in weight between the ages of 30-42 years, and increases by 2% for each additional pound between the ages of 50-62 years (Hubert, 1986).

The problems of obesity are not restricted to adults. There has been a significant rise in obesity among children (Dietz, 1994; Kotani et al., 1997). This earlier onset of obesity is likely due to a variety of factors including an increasingly sedentary lifestyle, and the prevalence of high fat convenience foods. However early onset obesity has consequences not only for the self-esteem of children and adolescents, but also leads to an increased risk of adult onset obesity with an accompanying increased risk of the myriad of obesity related disorders.

Obesity was once thought to be a side-effect of prosperity, or a result of the gluttony of Western culture. However, obesity is on the rise world-wide. Latin America, the Caribbean, Southeast Asia, and the Middle East have all experienced significant increases in the prevalence of obesity. For example, across Malaysia obesity is now more common than malnutrition (Popkin, 1994; Hodge, 1995; Forrester et al., 1996; Musaiger, 1996).

Due to the significant health impacts and accompanying human and monetary costs of obesity, methods for the prevention and treatment of this serious condition would be of significant value world wide.

Although the problems of obesity, or too much adipose tissue, are serious and have attracted a great deal of attention, fat in the body is not entirely bad. Adipose tissue is necessary to protect vital internal organs, to regulate energy balance and maintain homestasis, to maintain bone mass, and to support reproductive function. Conditions associated with too little adipose tissue include malnutrition, anorexia nervosa, and bulimia nervosa. Additionally, wasting is a condition often associated with other disease states including AIDS, cancer, and various cancer treatments. Such wasting has severe consequences for the quality of life of the patient, compromises the patient's ability to respond to treatment, and is often a major contributing factor to mortality. Therefore, methods for the treatment of conditions associated with lack of adipose tissue or a failure in adipocyte differentiation would be of significant value.

In addition to the consequences of obesity outlined above, obesity exacerbates or leads to a number of other serious health consequences. Therapies based on the inhibition of the growth, proliferation, differentiation, or survival of adipose tissue would help to ameliorate some of the secondary medical conditions caused by obesity and excess body fat.

One of the greatest dangers of obesity is that it significantly increases one's risk of developing type II diabetes. In the Nurses Cohort Study, body fatness was the dominant predictor for developing type II diabetes (Colditz et al., 1995).

Obesity leads to an increase in fasting plasma insulin, changes in glucose metabolism, and insulin resistance (Kolterman et al., 1980; Kopelman, 2000). For a period of time, the pancreatic β-cells are able to compensate for these obesity induced changes in glucose tolerance by producing more insulin. However, over time the pancreatic β-cells begin to fail leading to hyperglycemia similar to that observed in individuals with type I diabetes. The resulting increased blood glucose levels and lack of proper control of blood glucose place type II diabetics at risk for many of the same complications faced by type I diabetics including diabetic ketoacidosis, end-stage renal disease, diabetic retinopathy and amputation. There are also a host of less directly related conditions, such as hypertension, heart disease, peripheral vascular disease and infections, for which persons with diabetes are at substantially increased risk.

Increased body weight also has a substantial impact on cardiovascular function. Firstly, increased body weight results in an increase in total body oxygen consumption and an increase in blood volume. These changes lead to left ventricular alterations and increased stroke volume (De Divitiis, 1981; Licata, 1991). Over time, these changes in heart dynamics lead to changes in the size and shape of the heart resulting in cardiac hypertrophy. The consequences of cardiac hypertrophy and the other significant effects of obesity include an increased risk of congestive heart failure, hypertension, and coronary heart disease (Hubert et al., 1983; Willet 1995).

In addition to the serious consequence of obesity on cardiovascular function, obesity can lead to significant respiratory problems. Changes in the amount of fat tissue stored in the abdomen can alter the properties of the chest and diaphragm, and cause alterations in breathing patterns both during exertion and during rest. Ultimately, compromised breathing mechanics can lead to changes in arterial carbon dioxide levels, pulmonary hypertension, heart, and respiratory failure (Kopelman, 2000).

The consequences of obesity on respiration are perhaps most acutely observed when an individual is lying down, and thus are exacerbated during sleep (Kopelman, 1992; Grunstein, 1998). Obesity increases the frequency of sleep apnea, and can lead to severe hypoxia.

Additionally, sleep apnea often leads to poor sleep and the resultant daytime sleepiness can have serious consequences for daily activities including work and driving.

There appears to be a correlation between diet and obesity and some forms of cancer including colon cancer. However, in addition to this correlation between obesity and cancer, there are also several cancers of adipose and other soft tissue. These cancers are relatively rare, however they are often very difficult to detect. This is likely due to the fact that they usually grow for a relatively long time before causing pain; the soft tissue providing cushioning from surrounding nerves and hard tissue. Because they often go undetected for prolonged periods of time, such cancers frequently metastasize. Soft tissue and adipose cancers include liposarcoma, lipoma, hibernoma, and lipoblastoma (Lewandowski et al., 1996; Yoshikawa et al., 1999; Lakshmiah et al., 2000; Oliveira et al., 2001; Dilley et al., 2001).

Although the negative health consequences of obesity, and diseases aggravated by too much adipose tissue are well known, there are also a number of serious conditions associated with too little adipose tissue. Since adipose tissue is essential to protect internal organs and maintain homeostasis, conditions characterized by too little adipose tissue can be potentially fatal. The following illustrative examples of conditions characterized by too little adipose tissue highlight the need for treatments that enhance the growth, proliferation, differentiation, or survival of adipose tissue in such individuals.

Malnutrition is still a problem in both the developing and the developed world. Individuals suffering from malnutrition are susceptible to hypothermia and internal injury, as well as a host of vitamin and mineral deficiencies. For example, children lacking vitamin A can suffer impaired vision or even blindness.

Low birth weight is a serious problem world wide. Low birth weight can have many causes including poor pre-natal nutrition, in utero exposure to drugs and alcohol, and shortened gestation time. The consequences of low birth weight are severe, and the prognosis for low birth weight babies is compromised in comparison to normal birth weight babies. Complications associated with low birth weight can lead to long-term and even lifelong medical problems.

Anorexia nervosa and bulimia nervosa are two psychiatric conditions that result in a very low body weight and severe reduction in body fat. In some cases, the reduction in body fat in so severe that it results in loss of menses in adolescent girls, and loss of bone mass similar to that observed in menopausal women. These changes underscore the critical role played by adipose tissue in maintaining proper homeostasis including hormonal levels.

Finally, many diseases such as cancer and AIDS, as well as many cancer treatments, cause severe loss of both muscle and adipose tissue known as wasting. Wasting leads to loss of energy and vitality, and can severely inhibit a patient's ability to tolerate and recover from treatment. Additionally, the loss of energy and vitality has serious consequences for the quality of life of patients with these and other life threatening diseases.

### SUMMARY OF THE INVENTION

The present invention relates to the identification of secreted proteins differentially expressed between preadipocytes and adipocytes. Although the identified proteins are not novel, per se, their role in adipogenesis had not been previously appreciated.

The study of cell and developmental biology has often focused on the expression of genes in various cells and tissue types. Expression of messenger RNA, the product of transcription of the genetic material, can be measured by numerous methods well known in the art including Northern blot analysis, RT-PCR, and in situ hybridization. Such methods provide an incredible amount of detail concerning the expression of messenger RNA in a cell or tissue. More recently several techniques have been developed to allow one to make direct comparisons of the expression of RNA between tissues. These methods include differential display and subtractive hybridization analysis, and have added greatly to our understanding of the genetic mechanisms governing the development of many tissues and organs. Briefly, these methods allow one to identify the messages that are differentially expressed between two cell populations, and thus to hopefully pinpoint the basis of why two cell populations possess different characteristics while ignoring the countless number of genes that are similarly expressed between the two cell populations.

However, despite the tremendous amount of information gained from this type of analysis, the picture of the mechanisms of gene regulation and development that it offers is incomplete. These genomics based techniques provide no information about protein expression in cells or about differences in protein expression between two cell populations. It has long been appreciated that many genes are regulated post transcriptionally, and many of these modes of post-transcriptional regulation allow changes in the expression of proteins without altering the messenger RNA. Examples of such post-transcriptional regulation included phosphorylation, glycosylation, and cleavage of the pro-form of a protein to generate a functional isoform. It is unclear what percentage of genes are regulated entirely or in part by post-transcriptional mechanisms, and in fact it is quite likely that since so much of the study of gene regulation has been based on the analysis of messenger RNA, the frequency of post-transcriptional regulation of gene expression has been severely under-appreciated. For these reasons, a complete understanding of the differences between cell populations must include a proteomics based approach that can detect differences in protein expression between two cell populations even when such differences in protein expression occur independently of changes in the messenger RNA.

The present invention is directed to the discovery of proteins that are differentially expressed during the process of adipogenesis. The identification of proteins expressed either in pre-adipocytes or in adipocytes increases our understanding of the cellular and biochemical changes underlying this developmental process. Additionally, the identification of proteins involved in maintaining either the preadipogenic or the adipogenic state presents opportunities for detecting and treating the wide range of conditions characterized by hyper or hypo-adipogenesis.

One aspect of the invention provides proteomics based methods for identifying proteins involved in adipogenesis. Specifically, the invention provides a method for identifying a protein differentially expressed between a first and a second populations of cells of adipose lineage, comprising: (i) obtaining a first protein sample from said first populations of cells, and a second protein sample from said second populations of cells; (ii) separating proteins in said first and second protein samples; (iii) identifying and isolating one or more proteins, if any, differentially expressed in said first and said second populations of cells; and (iv) determining, by mass spectrometry, the identity/sequence of said differentially expressed proteins isolated in (iii).

In a related aspect, the invention provides a method for quantitating a protein differentially expressed between a first and a second populations of cells of adipose lineage, comprising: (i) obtaining a first protein sample from said first populations of cells, and a second protein sample from said second populations of cells; (ii) separating proteins in said first and second protein samples; (iii) identifying and isolating one or more proteins, if any, differentially expressed in said first and said second populations of cells; and (iv) determining, by mass spectrometry, the identity and relative quantity of said differentially expressed proteins isolated in (iii).

In one embodiment, protein samples of step (i) comprise secreted proteins.

In one embodiment, said first and said second populations of cells are each independently of embryonic, post-natal, or adult origin.

In one embodiment, both said first and said second populations of cells are derived from mammalian species, such as non-human primate or human.

In one embodiment, step (ii) can be effectuated by SDS-PAGE, or by nono-Liquid Chromatography coupled directly to mass spectrometer (nLC-MS).

In one embodiment, said first and second protein samples obtained in step (i) are digested before separation in step (ii).

In another aspect, the invention provides a method to identify and quantify the relative amounts of the proteins involved in adipogenesis. In a preferred embodiment, the proteins are isolated from either pre-adipocytes or differentiated adipocytes, and digested in solution to generate peptide fragments. The resulting peptide fragments are subjected to liquid chromatography (LC) and subsequently sequenced by mass spectrometry.

In a preferred embodiment, the sequence of the peptide fragments obtained from mass spectrometry is compared to a protein or nucleic acid database to identify sequence entries in the database which correspond to the differentially expressed proteins.

In one embodiment, the pre-adipocyte and adipocyte cell populations are both of embryonic origin.

In another embodiment, the two cell populations are both of post-natal origin.

In another embodiment, the two cell populations are both of adult origin.

In another embodiment, the origin of the two cell populations is independently selected from the group consisting of embryonic, post-natal, and adult.

In another embodiment, the two cell populations are both derived from a mammalian species. Preferrably the mammalian species is a non-human primate. Most preferably the mammalian species is human.

In one embodiment, when step (ii) is effectuated by SDS-PAGE, proteins identified and isolated in step (iii) is digested by in-gel digestion.

In one embodiment, proteins are identified as differentially expressed based on quantitation or semi-quantitation of separated proteins. Preferably, said quantitation or semi-quantitation is carried out by visual comparison of data generated in experimental and control samples. These data could include quantitative or semi-quantitative normalized mass spectrometry data and/or SDS-PAGE data.

In one embodiment, step (iv) is effected by tandem mass spectrometry (MS/MS).

In one embodiment, the method involves the identification of proteins differentially expressed in pre-adipocytes and in differentiated adipocytes. The proteins are separated by gel electrophoresis (non-limiting examples include one-dimensional and two-dimensional gel electrophoresis), bands corresponding to differentially expressed proteins are identified and excised, these bands are digested into peptide fragments, and the digested peptide fragments are subjected to mass spectrometry (non-limiting examples include nanospray mass spectrometry). From mass spectrometry, sequence data is obtained for the proteins present in the isolated bands. In a preferred embodiment, the resulting sequence data obtained from mass spectrometry is compared to a protein or nucleic acid database to identify sequence entries in the database which correspond to the differentially expressed proteins.

In one embodiment, the pre-adipocyte and adipocyte cell populations are both of embryonic origin.

In another embodiment, the two cell populations are both of post-natal origin.

In another embodiment, the two cell populations are both of adult origin.

In another embodiment, the origin of the two cell populations is independently selected from the group consisting of embryonic, post-natal, and adult.

In another embodiment, the two cell populations are both derived from a mammalian species. Preferably the mammalian species is a non-human primate. Most preferably the mammalian species is human.

In a second aspect, the invention provides a method to identify proteins involved in adipogenesis. The proteins are isolated from either pre-adipocytes or differentiated adipocytes, and digested in solution to generate peptide fragments. The resulting peptide fragments are subjected to liquid chromatography and subsequently sequenced by mass spectrometry. In a preferred embodiment, the sequence of the peptide fragments obtained from mass spectrometry is compared to a protein or nucleic acid database to identify sequence entries in the database which correspond to the differentially expressed proteins.

In one embodiment, the pre-adipocyte and adipocyte cell populations are both of embryonic origin.

In another embodiment, the two cell populations are both of post-natal origin.

In another embodiment, the two cell populations are both of adult origin.

In another embodiment, the origin of the two cell populations is independently selected from the group consisting of embryonic, post-natal, and adult.

In another embodiment, the two cell populations are both derived from a mammalian species. Preferably the mammalian species is a non-human primate. Most preferably the mammalian species is human.

In a third aspect, the invention provides the following proteins differentially expressed during adipogenesis: pigment epithelium derived factor, haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein and calgizzarin. These proteins had been previously identified, but their role in adipogenesis had not been envisioned.

In one embodiment, these differentially expressed proteins were identified by at least one of the proteomics based methods herein described.

In a second embodiment, the differentially expressed proteins were identified by comparing the sequence obtained by mass spectrometry to a protein or nucleic acid database.

The differential expression of factors between preadipocytes and adipocytes is suggestive of a role in adipogenesis.

In a fourth aspect, the invention provides a method for identifying factors differentially expressed during adipogenesis which are capable of affecting the proliferation, differentiation, or survival of cells. In certain embodiments, these cells are selected from pre-adipocytes, adipocytes, embryonic stem cells, adult stem cells, and fibroblasts.

In a preferred embodiment, factors which promote or enhance the growth, proliferation, differentiation, or survival of cells are adipogenic agonists.

In another preferred embodiment, factors which decrease or abrogate growth, proliferation, differentiation, or survival of cells are adipogenic antagonists.

In another preferred embodiment the adipogenic agonist or antagonist is a differentially expressed factor selected from: pigment epithelium derived factor, haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

In a preferred embodiment, the adipogenic agonist or antagonist is combined with a pharmaceutically acceptable carrier or excipient.

The invention also provides a method for identifying an agent capable of modulating adipogenesis, comprising: (i) identifying, using any of the methods described above, one or more proteins differentially expressed between pre-adipocytes and adipocytes; (ii) contacting cells in culture with said protein(s), wherein said cells are: preadipocytes, adipocytes, fibroblasts, embryonic stem cells, or adult stem cells; and, (iii) analyzing the cells in culture for changes in proliferation, differentiation, survival, or expression of adipogenesis marker genes, wherein a change in proliferation, differentiation, survival, or expression of adipogenesis marker genes after contacting said cells with said protein(s) indicates that said protein(s) is an agent capable of modulating adipogenesis.

In one embodiment, said agent is an adipogenic agonist which increases or potentiates the growth, proliferation, differentiation, or survival of said cells.

In one embodiment, said agent is an adipogenic antagonist which decreases or inhibits the growth, proliferation, differentiation, or survival of said cells.

In one embodiment, said one or more proteins differentially expressed between pre-adipocytes and adipocytes is selected from: pigment epithelium derived factor (PEDF), haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

In one embodiment, the method further comprises formulating said agent with a pharmaceutically acceptable carrier or excipient.

In a fifth aspect, the invention contemplates contacting a cell with an effective amount of an adipogenic agonist or antagonist, as identified above, to either increase or decrease adipogenesis in that cell. In preferred embodiments, the cell is contacted with a factor selected from: pigment epithelium derived factor, haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

In a related aspect, the invention provides a method for increasing adipogenesis in a cell, comprising contacting said cell with an effective amount of an adipogenic agonist.

In a related aspect, the invention provides a method for decreasing adipogenesis in a cell, comprising contacting said cell with an effective amount of an adipogenic antagonist.

In a sixth aspect, the invention contemplates administering an effective amount of an adipogenic agonist or antagonist, as identified above, to a patient having a condition characterized by hyper or hypo-adipogenesis.

In a preferred embodiment, the patient is a human patient. In a preferred embodiment, the condition is selected from: malnutrition, anorexia nervosa, bulimia nervosa, low birth weight, wasting associated with AIDS, cancer, or side effects of cancer therapy. In a related embodiment, the patient is a fetus and the adipogenic agonist is administered in utero.

In another preferred embodiment, the adipogenic factor is selected from: pigment epithelium derived factor, haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

In one embodiment, the patient has a condition characterized by hyper-adipogenesis and the condition is treated by administering an effective amount of an adipogenic antagonist. In a preferred embodiment, the hyper-adipogenic condition is selected from the group consisting of obesity, hyperlipidemia, hypercholesterolemia, hypertriglyceridemia, liposarcoma, lipoma, hibernoma, and lipoblastoma.

In a second embodiment, the patient has a condition exacerbated by hyper-adipogenic conditions such as obesity, and the condition is treated by administering an effective amount of an adipogenic antagonist. In a preferred embodiment, the condition exacerbated by the hyper-adipogenic condition is selected from the group consisting of type II diabetes, high blood pressure, osteoarthritis, asthma, respiratory insufficiency, coronary heart disease, cancer, and sleep apnea.

In another embodiment, the patient has a condition characterized by hypo-adipogenesis and the condition is treated by administering an effective amount of an adipogenic agonist. In a preferred embodiment, the hypo-adipogenic condition is selected from the group consisting of malnutrition, anorexia nervosa, bulimia nervosa, low birth weight, and wasting associated with AIDS, cancer, and the side effects of cancer therapy.

In another embodiment, the patient is an animal. Preferably the animal is a farm animal, and most preferably the animal is a farm animal selected from the group consisting of cows, pigs, sheep, chickens, ducks, goats, deer, and buffalo. In a preferred embodiment, the adipogenic agonist is administered to increase the size and/or fat content of the animal.

In a related aspect, the invention provides a method of modulating adipogenesis in a cell, comprising contacting the cell with an effective amount of an agent selected from: pigment epithelium derived factor (PEDF), haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

In a related aspect, the invention provides a method of determinig the differentiation stage of adipogenesis in a cell, comprising identifying one or more agents whose expression level is substantially changed during adipogenesis, and determining the expression level of said one or more agents during adipogenesis of said cell, thereby determining the differentiation stage of adipogenesis in said cell.

In a seventh aspect, the invention also contemplates an expression cassette comprising a transcriptional initiation region, a nucleic acid sequence encoding an adipogenic factor under the transcriptional regulation of said transcriptional initiation region, and a transcriptional termination region. Preferably the nucleic acid sequence encoding the adipogenic factor is selected from: pigment epithelium derived factor, haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

In one embodiment, the transcriptional initiation region is heterologous to the nucleic acid sequence.

In another embodiment, the transcriptional initiation region is homologous to the nucleic acid sequence.

In an eighth aspect, the invention includes a cell comprising an expression cassette comprising a transcriptional initiation region consisting of a 5′ non-coding region regulating the transcription of a nucleic acid sequence encoding an adipogenic factor, a promoter and enhancer, a marker gene, and a transcriptional termination region. Preferably the nucleic acid sequence encoding the adipogenic factor is selected from: pigment epithelium derived factor, haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

In a preferred embodiment, the invention includes a method for producing and purifying an adipogenic factor by culturing the cell comprising this expression cassette. In another preferred embodiment, the invention includes the purified culture of cells expressing said expression cassette.

In a ninth aspect, the invention includes methods of treating a patient using cells comprising the expression cassette comprising an adipogenic factor or factors. Preferably the cells contain a nucleic acid sequence encoding an adipogenic factor selected from: pigment epithelium derived factor, haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

In a preferred embodiment, the patient is a human.

In one embodiment, the patient is suffering from a condition characterized by hyper-adipogenesis, and is treated with an effective amount of cells expressing an adipogenic antagonist. In a preferred embodiment, the hyper-adipogenic condition is selected from the group consisting of obesity, hyperlipidemia, hypercholesterolemia, hypertriglyceridemia, liposarcoma, lipoma, hibernoma, and lipoblastoma.

In a second embodiment, the patient is suffering from a condition exacerbated by excess body fat, and is treated with an effective amount of cells expressing an adipogenic antagonist. In a preferred embodiment, the condition exacerbated by excess body fat or obesity is selected from the group consisting of type II diabetes, high blood pressure, osteoarthritis, asthma, respiratory insufficiency, coronary heart disease, cancer, and sleep apnea.

In a third embodiment, the patient is suffering from a condition characterized by hypo-adipogenesis, and is treated with an effective amount of cells expressing an adipogenic agonist. In a preferred embodiment, the hypo-adipogenic condition is selected from the group consisting of malnutrition, anorexia nervosa, bulimia nervosa, low birth weight, and wasting associated with AIDS, cancer, and the side effects of cancer therapy.

In a fourth embodiment, the patient is an animal. Preferably a farm animal. Most preferably a farm animal selected from the group consisting of cows, pigs, sheep, chickens, ducks, goats, deer, and buffalo. The invention comprises administering to the animal an effective amount of cells expressing an adipogenic agonist sufficient to increase the size and/or fat content of the animal.

In a tenth aspect, the invention contemplates a method for conducting a weight loss business comprising identifying and recruiting clients in need of weight reduction for personal and/or medical reasons, providing these clients with a treatment regimen which includes an effective amount of an adipogenic antagonist identified using any of the suitable methods of the instant invention, monitoring the weight of these clients over time, and adjusting the treatment regimen in lieu of the change in weight over time.

In a preferred embodiment, the weight loss business includes a system for billing the client or the client's insurance carrier.

In another preferred embodiment, the weight loss business includes a marketing, advertising, and sales force.

A related aspect provides a method of conducting a drug discovery business comprising: (i) identifying, using the method of claim x, one or more agents capable of modulating adipogenesis; (ii) conducting therapeutic profiling of agents identified in step (i), or further analogs thereof, for efficacy and toxicity in animals; and (iii) formulating a pharmaceutical preparation including one or more agents identified in step (ii) as having an acceptable therapeutic profile.

In one embodiment, the method further comprises a step of establishing a distribution system for distributing the pharmaceutical preparation for sale.

In one embodiment, the method further comprises a step of establishing a sales group for marketing the pharmaceutical preparation.

A related aspect provides a method of conducting a target discovery business comprising: (i) identifying, using the method of claim x, one or more agents capable of modulating adipogenesis; (ii) (optionally) conducting therapeutic profiling of agents identified in step (i) for efficacy and toxicity in animals; and (iii) licensing, to a third party, the rights for further drug development and/or sales for gents identified in step (i), or analogs thereof.

In an eleventh aspect, the invention contemplates a method for identifying small molecules that modulate the activity of an adipogenic agonist or antagonist. The invention further contemplates the small molecule which modulates that activity of an adipogenic agonist or an adipogenic antagonist. Preferably, the small molecule can be used to treat a patient.

In one embodiment, the patient is suffering from a condition characterized by hyper-adipogenesis, and is treated with an effective amount of cells expressing an adipogenic antagonist. In a preferred embodiment, the hyper-adipogenic condition is selected from the group consisting of obesity, hyperlipidemia, hypercholesterolemia, hypertriglyceridemia, liposarcoma, lipoma, hibernoma, and lipoblastoma.

In a second embodiment, the patient is suffering from a condition exacerbated by excess body fat, and is treated with an effective amount of cells expressing an adipogenic antagonist. In a preferred embodiment, the condition exacerbated by excess body fat or obesity is selected from the group consisting of type II diabetes, high blood pressure, osteoarthritis, asthma, respiratory insufficiency, coronary heart disease, cancer, and sleep apnea.

In a third embodiment, the patient is suffering from a condition characterized by hypo-adipogenesis, and is treated with an effective amount of cells expressing an adipogenic agonist. In a preferred embodiment, the hypo-adipogenic condition is selected from the group consisting of malnutrition, anorexia nervosa, bulimia nervosa, low birth weight, and wasting associated with AIDS, cancer, and the side effects of cancer therapy.

In a fourth embodiment, the patient is an animal. Preferably a farm animal. Most preferably a farm animal selected from the group consisting of cows, pigs, sheep, chickens, ducks, goats, deer, and buffalo. The invention comprises administering to the animal an effective amount of cells expressing an adipogenic agonist sufficient to increase the size and/or fat content of the animal.

The practice of the present invention will employ, unless otherwise indicated, conventional techniques of cell biology, cell culture, molecular biology, transgenic biology, microbiology, recombinant DNA, and immunology, which- are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### DESCRIPTION OF THE DRAWINGS

FIG. 1 illustrates the metabolic labeling of proteins secreted as 3T3-L1 cells are progressing from preadipocytes to differentiated adipocytes. Proteins were labeled by culturing cells in the presence of ³⁵S-methionine. The cell culture supernatants were harvested and resolved on either 7% (A) or 15% (B) SDS-poly acrylimide gels (SDS-PAGE) followed by autoradiography.

FIG. 2 illustrates proteins secreted by either pre-adipocytes or day 9 differentiated adipocytes. Cell culture supernatants were harvested and resolved on either 7% (A) or 15% (B) SDS-PAGE gels and then visualized by silver-staining. Numbered arrows indicate the bands corresponding to differentially expressed proteins that were excised and later analyzed by mass spectrometry.

FIG. 3 shows the analysis of differentially expressed protein bands identified in FIG. 2. The bands were excised, subjected to in-gel digestion, and analyzed by tandem mass spectrometry. (A) The spectrum from MS/MS analysis of protein band 8 corresponding to PEDF. (B) The spectrum from MS/MS analysis of protein band 12 corresponding to haptoglobin. (C) The spectrum from MS/MS analysis of protein band 16 corresponding to NGAL. (D) The spectrum from MS/MS analysis of protein band 16 corresponding to HCNP. The y series of ions (C-terminal fragments) as well as those from the b series (N-terminal fragments) are shown. The sequence of the peptides as deduced from the spectrum and database search are given at the top of each panel.

FIG. 4 shows an analysis of the mRNA of the identified differentially expressed secreted proteins. (A) Illustrates the results of RT-PCR analysis performed on RNA isolated from preadipocytes and day 9 adipocytes using primers specific for PEDF, haptoglobin, NGAL, HCNP, adipsin, and Acrp30. (B) Illustrates the results of Northern blot analysis of RNA isolated from preadipocytes and day 9 adipocytes using probes specific for PEDF and haptoglobin.

Table 1 lists the secreted proteins identified by nanoelectrospray tandem mass spectrometry, and the band number from which each protein was isolated. Three of the identified proteins were down regulated and eight were up-regulated between preadipocytes and day 9 adipocytes.

Table 2 lists the twelve additional secreted proteins identified by LC-MS/MS.

Table 3 lists the relative quantitation of proteins secreted by preadipocytes and adipocytes using SILAC methodology.

### DETAILED DESCRIPTION OF THE INVENTION

### 1. Overview

The following secreted proteins were identified in a screen for factors differentially expressed between preadipocytes and adipocytes. The proteins themselves may or may not be novel, however their role in adipogenesis had not been previously appreciated. Therefore, the present invention discloses previously unrecognized functions for these proteins in adipogenesis. The identification of proteins differentially expressed during adipogenesis has implications not only for the study of developmental biology, but also for the treatment of diseases and conditions related to hyper or hypo adipogenesis.

Despite the societal implications surrounding too much adipose tissue, fat is absolutely essential for living organisms. Adipose tissue is important in protecting and cushioning our vital internal organs, helps to regulate body temperature and maintain homeostasis, and regulates satiety. Although a wide range of conditions are associated with too much adipose tissue, there are also a range of health consequences associated with too little adipose tissue. The identification of factors expressed differentially between preadipocytes and adipocytes not only increases our understanding of the biological process of adipogenesis, but also provides potential therapeutic means for either stimulating or suppressing adipogenesis.

The present invention relates to the use of two proteomics based approaches to identify factors, especially secreted factors differentially expressed during adipogenesis. Using these two methods, we have identified several secreted proteins including both proteins previously recognized for an involvement in adipogenesis and proteins whose role in adipogenesis was not previously appreciated. Some of these factors are expressed preferentially in preadipocytes while others are expressed preferentially in adipocytes. The invention contemplates, among other things, methods for identifying differentially expressed proteins during adipogenesis, the use of these factors to regulate the level of adipogenesis in a cell, the use of these factors to treat conditions associated with too much or too little adipose tissue, and expression cassettes and cells expressing such adipogenic factors.

The application of factors involved in adipogenesis to the problem of hyper-adipogenic differentiation has been previously described. The most prominent model for such an approach concerns the use of the protein leptin (reviewed Auwerx and Staels, 1998; Friedman and Halaas, 1998; Dagogo-Jack, 2001). Leptin is a 167 amino acid secreted protein transcribed from the ob gene, and originally identified in ob/ob obesity prone mice. Leptin is produced by white adipose tissue, and an approximately 16 kD form circulates in the body.

In both wild type and ob/ob mice, injection or subcutaneous infusion of leptin results in a dose-dependent decrease in body fat (Halaas et al., 1995, Halaas et al., 1997). Treatment with leptin seems to stimulate weight loss via a number of mechanisms including the inhibition of food consumption, the stimulation of energy expenditure, and the amelioration of insulin resistance. More recently, human trials have demonstrated that treatment with leptin results in progressive weight loss in obese individuals with chronic leptin deficiency, as well as in non-obese individuals and obese individuals who apparently lack congenital leptin abnormalities.

Leptin is not the only factor expressed during adipogenesis that has been used to regulate fat reduction in human and animal subjects. Adipocyte complement related protein (Acrp 30) is a secreted protein of unknown function expressed in differentiated adipocytes (Scherer et al., 1995). The protein contains four main domain, and it has recently been demonstrated that treatment of mice with the globular head domain of Acrp 30 results in weight reduction. This weight reduction occurred despite the consumption of a high fat/high-sucrose diet, and did not appear to be due to a decrease in caloric intake (Fruebis et al., 2001).

The present invention describes the identification and characterization of several additional factors involved in adipogenesis that can be used therapeutically to modulate adipogenesis in vitro or in vivo. We describe here two proteomics based approaches used to identify secreted factors differentially expressed during adipogenesis. The validity and effectiveness of this approach is confirmed by the identification of several proteins previously recognized for their role in adipogenesis. These factors include fibronectin, procollagen type I α2, adipocyte complement-related protein 30 kDa (Acrp 30), complement factor C3 precursor, adipsin, entactin/nidogen, α3 subunit of type VI collagen, resistin, SPARC, and cystatin 3. Additionally, we have identified a number of proteins whose involvement in adipogenesis had not been previously appreciated. These factors represent previously unrecognized molecular components of the adipogenic process, and provide novel targets for therapeutic intervention to either promote or inhibit adipogenesis.

### 2. Definitions

For convenience, certain terms employed in the specification, examples, and appended claims are collected here.

“Agonist”, as used herein, refers to a factor which enhances or augments the growth, proliferation, or survival of a cell. In this context, such a factor would favor or promote adipogenesis.

“Antagonist”, as used herein, refers to a factor which decreases the growth, proliferation, or survival of a cell. In this context, such a factor would favor or promote a pre-adipogenic state, and would disfavor the differentiation of adipocytes.

The term “differentially expressed” refers generally to either mRNA or protein that is present in one cell or tissue but is either not expressed in another cell or tissue, or is expressed in the other cell or tissue at a substantially different level or in a different form. Preferably, if the normalized expression level of a certain protein in a first sample is at least about 20%, 40%, 50%, 60%, 80%, 100% (1-fold), 2-fold, 3-fold, 4-fold, 5-fold, 10-fold, 20-fold, 50-fold, 100-fold, or 200-fold or more when compared to its expression in a second sample, the protein is considered differentially expressed in the two samples. Either the first or the second sample can be a control/untreated/normal sample, while the other is a experimental/treated/disease sample. For example, if protein X is expressed in skeletal muscle but not in cardiac muscle, then protein X is differentially expressed. Similarly, if protein X is expressed in a hyper-phosphorylated form in skeletal muscle and a hypo-phosphorylated form in cardiac muscle, then protein X is differentially expressed. The methods described herein are based specifically upon the identification of factors that are differentially expressed at the protein level. Such factors may or may not be differentially expressed at the mRNA level.

“Cells of adipose lineage” refers to cells committed to differentiate into adipose tissue. It includes cells in all stages of the adipogenesis (differentiation) process. Representative cells of adipose lineage includes (but are not limited to) preadipocytes, and adipocytes.

“Preadipocyte” refers to a cell that has the potential to differentiate according to an adipogenic program, but has not yet done so.

“Adipocyte” refers to a cell that has differentiated along an adipogenic program. Adipocytes can be identified according to characteristic marker gene expression, as well as characteristic changes in cell shape and the accumulation of lipids. Such marker genes (see below) are well-known in the art.

“Adipogenesis marker genes” refers to one or a set of genes specifically associated with a specific adipogenesis/differentiation stage. Such marker genes are well-known in the art. For example, differentiated adipocyte marker genes include, to name just a few, glycerophosphate dehydrogenase (GPDH), fatty acid synthase, acetyl CoA carboxylase, malic enzyme, Glut 4, the insulin receptor, and aP2 (the adipocyte-selective fatty acid binding protein) (see Spiegelman et al. J. Biol. Chem. 268: 6823-6826, 1993, incorporated herein by reference). Preadipocytes also have characteristic marker genes, such as the cell surface antigen recognized by the monoclonal antibody AD-3. Expression level changes of the various isoforms of the C/EBP (CCAAT/enhancer-binding proteins) family of transcription factors may also indicate different stages of adipogenesis (see Yu and Hausman, Exp Cell Res Dec. 15, 1998; 245(2): 343-9).

“Adipogenesis” refers to the process whereby adipose tissue, a mesodermal derivative, develops from preadipocytes

“Proliferation” refers to an increase in cell number. Proliferation can be measured by many commonly employed techniques including BrdU labeling and incorporation of tritiated hydrogen (³H).

“Growth” refers to an increase in cell size.

“Differentiation” refers to the formation of cells expressing markers known to be associated with cells that are more specialized and closer to becoming terminally differentiated cells incapable of further division or differentiation.

“Survival” refers to the characteristic of being alive. In a cellular context, it is the opposite of conditions of cell death such as apoptosis and necrosis. As used herein, a factor which enhances cellular survival can do so either by increasing survival or by decreasing cell death.

An “effective amount” of, e.g., an adipogenic factor refers to an amount of a factor which is sufficient to bring about a change in the rate of cell growth or proliferation, and/or the state of differentiation, and/or the state of survival of a cell.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding a polypeptide and comprising exon coding sequences, though it may optionally include intron sequences derived from a chromosomal gene. The term “intron” refers to a DNA sequence present in a given gene which is not translated into protein and is generally found between exons.

As used herein, the term “nucleic acid” refers to polynucleotides such as deoxyribonucleic acid (DNA), and where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

### 3. Detailed Description of the Invention

A. Adipocyte Cultures and Adipogenesis

The formation of adipose tissue, or adipogenesis, is an important developmental process (Rosen et al., 2000). Our recent understanding of this process has been greatly aided by the establishment of immortal preadipocyte cell lines that provide an experimentally accessible system in vitro, many features of which faithfully recapitulate this process in vivo. These features include morphological changes, cessation of cell growth, expression of many lipogenic enzymes, extensive lipid accumulation, and the establishment of sensitivity to most or all of the key hormones that impact on this cell type, including insulin.

In the 1970's, Green and his colleagues first established several immortal fibroblast lines that readily differentiated into adipocytes when appropriate hormonal inducers were added (Green and Kehinde Cell 1: 113-116, 1974; Cell 5: 19-27, 1975; Cell 7: 105-113, 1976). These lines, designated 3T3-L1 and 3T3-F442A, were isolated from nonclonal Swiss 3T3 cells and are already committed (or determined) to the adipocytic lineage. When treated with an empirically-derived prodifferentiative regimen that includes cAMP, insulin, and glucocorticoids, they undergo differentiation to mature fat cells over a 4-6 day period. Subsequently, committed preadipocyte lines have been derived independently by others (Negrel et al. Proc. Natl. Acad. Sci. 75: 6054-6058, 1978; Chapman et al. J. Biol. Chem. 259: 15548-15555, 1984); despite minor differences in their optimal differentiation conditions they behave very similarly to 3T3-L1 and 3T3-F442A cells. More recently, studies have been performed using pluripotent stem cells that can be induced to yield adipose tissue in addition to several other lineages. For example, marrow-derived stromal cells can be induced to form both bone cells and fat cells (Pittenger et al. Science 284: 143-147, 1999). Although stem cell technology is developing rapidly, their recent introduction and the complexity inherent in these systems has prevented them from being extensively used in the adipogenesis field. Almost all work on adipogenesis has utilized either the aforementioned predetermined clonal cell lines or cultured preadipocytes isolated from the stromal-vascular fraction of dissociated fat pads.

Adipogenesis in vitro follows a highly ordered and well characterized temporal sequence. Initially, there is growth arrest of proliferating preadipocytes, usually achieved in cultured cell lines after contact inhibition. In cultured cell models, initial growth arrest is induced by the addition of a prodifferentiative hormonal regimen and is followed by one or two additional rounds of cell division known as clonal expansion. This process ceases coincident with the expression of the key transcription factors peroxisome proliferator-activated receptor γ (PPARγ) and CCAAT/enhancer binding protein α (C/EBPα). The induction of these two proteins is characterized by a second, permanent period of growth arrest followed by expression of the fully differentiated phenotype. The exact mechanisms by which PPARγ and C/EBPα bring about this change is unclear. In nonconfluent 3T3-F442A cells, activation of PPARγ correlates with loss of DNA binding activity of E2F/DP, a central transcriptional player in the regulation of many genes involved in cell growth. This alteration in E2F/DP binding is secondary to a decrease in the protein phosphatase PP2A, which results in increased phosphorylation of DP-1, blocking DNA binding. The E2F family of transcription factors are known to promote cell division in a variety of models of cellular growth and differentiation.

The process of terminal differentiation occurs over several days in cultured cell lines. A second, permanent state of growth arrest occurs followed by the accumulation of phenotypic markers of the mature adipocyte. The earliest events include a morphological rounding up of the fibroblast-like preadipocytes and the expression of mRNAs including lipoprotein lipase and the transient induction of the transcriptional components C/EBPβ and C/EBPδ (MacDougald and Lane Annu. Rev. Biochem. 64: 345-373, 1995; Darlington et al. J. Biol. Chem. 273: 30057-30060, 1998). These earliest events are followed by the appearance of PPARγ and C/EBPα, which activate de novo or enhanced expression of most or all of the genes that characterize the adipocyte phenotype. These adipocyte marker genes include (to name just a few) glycerophosphate dehydrogenase (GPDH), fatty acid synthase, acetyl CoA carboxylase, malic enzyme, Glut 4, the insulin receptor, and aP2 (the adipocyte-selective fatty acid binding protein) (Spiegelman et al. J. Biol. Chem. 268: 6823-6826, 1993, incorporated herein by reference). Throughout this process, lipid-laden droplets begin to appear in the cytoplasm, and over time they become quite large and often coalesce into one or a few major droplets.

A number of transcription factors have been identified which play prominent roles in adipogenesis including PPARγ, C/ERP, and ADD1 (reviewed in Rosen et al., 2000). For example, PPARγ is required for adipocyte differentiation, and overexpression of PPARγ via retroviral infection can stimulate adipocyte differentiation in fibroblasts (Rosen et al., 1999). These studies demonstrate that factors involved in adipogenesis can be used to regulate this process in animals with possible therapeutic implications.

Although several non-limiting model systems for adipogenesis in vitro are described here, either as published literature or as disclosed specific experimental conditions, it should be understood that other known/accepted model systems for adipogenesis not explicitly described in the instant application can also be used or adapted to be used in practicing the instant invention.

B. Mass Spectrometers, Detection Methods and Sequence Analysis

In certain embodiments, the interacting proteins are identified by protease digestion followed by mass spectrometry. During the past decade, new techniques in mass spectrometry have made it possible to accurately measure with high sensitivity the molecular weight of peptides and intact proteins. These techniques have made it much easier to obtain accurate peptide masses of a protein for use in databases searches. Mass spectrometry provides a method, of protein identification that is both very sensitive (10 fmol-1 pmol) and very rapid when used in conjunction with sequence databases. Advances in protein and DNA sequencing technology are resulting in an exponential increase in the number of protein sequences available in databases. As the size of DNA and protein sequence databases grows, protein identification by correlative peptide mass matching has become an increasingly powerful method to identify and characterize proteins.

Mass Spectrometry

Mass spectrometry, also called mass spectroscopy, is an instrumental approach that allows for the gas phase generation of ions as well as their separation and detection. The five basic parts of any mass spectrometer include: a vacuum system; a sample introduction device; an ionization source; a mass analyzer; and an ion detector. A mass spectrometer determines the molecular weight of chemical compounds by ionizing, separating, and measuring molecular ions according to their mass-to-charge ratio (m/z). The ions are generated in the ionization source by inducing either the loss or the gain of a charge (e.g. electron ejection, protonation, or deprotonation). Once the ions are formed in the gas phase they can be electrostatically directed into a mass analyzer, separated according to mass and finally detected. The result of ionization, ion separation, and detection is a mass spectrum that can provide molecular weight or even structural information.

A common requirement of all mass spectrometers is a vacuum. A vacuum is necessary to permit ions to reach the detector without colliding with other gaseous molecules. Such collisions would reduce the resolution and sensitivity of the instrument by increasing the kinetic energy distribution of the ion's inducing fragmentation, or preventing the ions from reaching the detector. In general, maintaining a high vacuum is crucial to obtaining high quality spectra.

The sample inlet is the interface between the sample and the mass spectrometer. One approach to introducing sample is by placing a sample on a probe which is then inserted, usually through a vacuum lock, into the ionization region of the mass spectrometer. The sample can then be heated to facilitate thermal desorption or undergo any number of high-energy desorption processes used to achieve vaporization and ionization.

Capillary infusion is often used in sample introduction because it can efficiently introduce small quantities of a sample into a mass spectrometer without destroying the vacuum. Capillary columns are routinely used to interface the ionization source of a mass spectrometer with other separation techniques including gas chromatography (GC) and liquid chromatography (LC). Gas chromatography and liquid chromatography can serve to separate a solution into its different components prior to mass analysis. Prior to the 1980's, interfacing liquid chromatography with the available ionization techniques was unsuitable because of the low sample concentrations and relatively high flow rates of liquid chromatography. However, new ionization techniques such as electrospray were developed that now allow LC/MS to be routinely performed. One variation of the technique is that high performance liquid chromatography (HPLC) can now be directly coupled to mass spectrometer for integrated sample separation/preparation and mass spectrometer analysis.

In terms of sample ionization, two of the most recent techniques developed in the mid 1980's have had a significant impact on the capabilities of Mass Spectrometry: Electrospray Ionization (ESI) and Matrix Assisted Laser Desorption/Ionization (MALDI). ESI is the production of highly charged droplets which are treated with dry gas or heat to facilitate evaporation leaving the ions in the gas phase. MALDI uses a laser to desorb sample molecules from a solid or liquid matrix containing a highly UV-absorbing substance.

The MALDI-MS technique is based on the discovery in the late 1980s that an analyte consisting of, for example, large nonvolatile molecules such as proteins, embedded in a solid or crystalline “matrix” of laser light-absorbing molecules can be desorbed by laser irradiation and ionized from the solid phase into the gaseous or vapor phase, and accelerated as intact molecular ions towards a detector of a mass spectrometer. The “matrix” is typically a small organic acid mixed in solution with the analyte in a 10,000:1 molar ratio of matrix/analyte. The matrix solution can be adjusted to neutral pH before mixing with the analyte.

The MALDI ionization surface may be composed of an inert material or else modified to actively capture an analyte. For example, an analyte binding partner may be bound to the surface to selectively absorb a target analyte or the surface may be coated with a thin nitrocellulose film for nonselective binding to the analyte. The surface may also be used as a reaction zone upon which the analyte is chemically modified, e.g., CNBr degradation of protein. See Bai et al, Anal. Chem. 67, 1705-1710 (1995).

Metals such as gold, copper and stainless steel are typically used to form MALDI ionization surfaces. However, other commercially-available inert materials (e.g., glass, silica, nylon and other synthetic polymers, agarose and other carbohydrate polymers, and plastics) can be used where it is desired to use the surface as a capture region or reaction zone. The use of Nation and nitrocellulose-coated MALDI probes for on-probe purification of PCR-amplified gene sequences is described by Liu et al., Rapid Commun. Mass Spec. 9:735-743 (1995). Tang et al. have reported the attachment of purified oligonucleotides to beads, the tethering of beads to a probe element, and the use of this technique to capture a complimentary DNA sequence for analysis by MALDI-TOF MS (reported by K. Tang et al., at the May 1995 TOF-MS workshop, R. J. Cotter (Chairperson); K. Tang et al., Nucleic Acids Res. 23, 3126-3131, 1995). Alternatively, the MALDI surface may be electrically- or magnetically activated to capture charged analytes and analytes anchored to magnetic beads respectively.

Aside from MALDI, Electrospray Ionization Mass Spectrometry (ESI/MS) has been recognized as a significant tool used in the study of proteins, protein complexes and bio-molecules in general. ESI is a method of sample introduction for mass spectrometric analysis whereby ions are formed at atmospheric pressure and then introduced into a mass spectrometer using a special interface. Large organic molecules, of molecular weight over 10,000 Daltons, may be analyzed in a quadrupole mass spectrometer using ESI.

In ESI, a sample solution containing molecules of interest and a solvent is pumped into an electrospray chamber through a fine needle. An electrical potential of several kilovolts may be applied to the needle for generating a fine spray of charged droplets. The droplets may be sprayed at atmospheric pressure into a chamber containing a heated gas to vaporize the solvent. Alternatively, the needle may extend into an evacuated chamber, and the sprayed droplets are then heated in the evacuated chamber. The fine spray of highly charged droplets releases molecular ions as the droplets vaporize at atmospheric pressure. In either case, ions are focused into a beam, which is accelerated by an electric field, and then analyzed in a mass spectrometer.

Because electrospray ionization occurs directly from solution at atmospheric pressure, the ions formed in this process tend to be strongly solvated. To carry out meaningful mass measurements, solvent molecules attached to the ions should be efficiently removed, that is, the molecules of interest should be “desolvated.” Desolvation can, for example, be achieved by interacting the droplets and solvated ions with a strong countercurrent flow (6-9 l/m) of a heated gas before the ions enter into the vacuum of the mass analyzer.

Other well-known ionization methods may also be used. For example, electron ionization (also known as electron bombardment and electron impact), atmospheric pressure chemical ionization (APCI), fast atom Bombardment (FAB), or chemical ionization (CI).

Immediately following ionization, gas phase ions enter a region of the mass spectrometer known as the mass analyzer. The mass analyzer is used to separate ions within a selected range of mass to charge ratios. This is an important part of the instrument because it plays a large role in the instrument's accuracy and mass range. Ions are typically separated by magnetic fields, electric fields, and/or measurement of the time an ion takes to travel a fixed distance.

If all ions with the same charge enter a magnetic field with identical kinetic energies a definite velocity will be associated with each mass and the radius will depend on the mass. Thus a magnetic field can be used to separate a monoenergetic ion beam into its various mass components. Magnetic fields will also cause ions to form fragment ions. If there is no kinetic energy of separation of the fragments the two fragments will continue along the direction of motion with unchanged velocity. Generally, some kinetic energy is lost during the fragmentation process creating non-integer mass peak signals which can be easily identified. Thus, the action of the magnetic field on fragmented ions can be used to give information on the individual fragmentation processes taking place in the mass spectrometer.

Electrostatic fields exert radial forces on ions attracting them towards a common center. The radius of an ion's trajectory will be proportional to the ion's kinetic energy as it travels through the electrostatic field. Thus an electric field can be used to separate ions by selecting for ions that travel within a specific range of radii which is based on the kinetic energy and is also proportion to the mass of each ion.

Quadrupole mass analyzers have been used in conjunction with electron ionization sources since the 1950s. Quadrupoles are four precisely parallel rods with a direct current (DC) voltage and a superimposed radio-frequency (RF) potential. The field on the quadrupoles determines which ions are allowed to reach the detector. The quadrupoles thus function as a mass filter. As the field is imposed, ions moving into this field region will oscillate depending on their mass-to-charge ratio and, depending on the radio frequency field, only ions of a particular m/z can pass through the filter. The m/z of an ion is therefore determined by correlating the field applied to the quadrupoles with the ion reaching the detector. A mass spectrum can be obtained by scanning the RF field. Only ions of a particular m/z are allowed to pass through.

Electron ionization coupled with quadrupole mass analyzers can be employed in practicing the instant invention. Quadrupole mass analyzers have found new utility in their capacity to interface with electrospray ionization. This interface has three primary advantages. First, quadrupoles are tolerant of relatively poor vacuums (˜5×10⁻⁵ torr), which makes it well-suited to electrospray ionization since the ions are produced under atmospheric pressure conditions. Secondly, quadrupoles are now capable of routinely analyzing up to an m/z of 3000, which is useful because electrospray ionization of proteins and other biomolecules commonly produces a charge distribution below m/z 3000. Finally, the relatively low cost of quadrupole mass spectrometers makes them attractive as electrospray analyzers.

The ion trap mass analyzer was conceived of at the same time as the quadrupole mass analyzer. The physics behind both of these analyzers is very similar. In an ion trap the ions are trapped in a radio frequency quadrupole field. One method of using an ion trap for mass spectrometry is to generate ions externally with ESI or MALDI, using ion optics for sample injection into the trapping volume. The quadrupole ion trap typically consist of a ring electrode and two hyperbolic endcap electrodes. The motion of the ions trapped by the electric field resulting from the application of RF and DC voltages allows ions to be trapped or ejected from the ion trap. In the normal mode the RF is scanned to higher voltages, the trapped ions with the lowest m/z and are ejected through small holes in the endcap to a detector (a mass spectrum is obtained by resonantly exciting the ions and thereby ejecting from the trap and detecting them). As the RF is scanned further, higher m/z ratios become are ejected and detected. It is also possible to isolate one ion species by ejecting all others from the trap. The isolated ions can subsequently be fragmented by collisional activation and the fragments detected. The primary advantages of quadrupole ion traps is that multiple collision-induced dissociation experiments can be performed without having multiple analyzers. Other important advantages include its compact size, and the ability to trap and accumulate ions to increase the signal-to-noise ratio of a measurement.

Quadrupole ion traps can be used in conjunction with electrospray ionization MS/MS experiments in the instant invention.

The earliest mass analyzers separated ions with a magnetic field. In magnetic analysis, the ions are accelerated (using an electric field) and are passed into a magnetic field. A charged particle traveling at high speed passing through a magnetic field will experience a force, and travel in a circular motion with a radius depending upon the m/z and speed of the ion. A magnetic analyzer separates ions according to their radii of curvature, and therefore only ions of a given m/z will be able to reach a point detector at any given magnetic field. A primary limitation of typical magnetic analyzers is their relatively low resolution.

In order to improve resolution, single-sector magnetic instruments have been replaced with double-sector instruments by combining the magnetic mass analyzer with an electrostatic analyzer. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument to 100,000. Magnetic double-focusing instrumentation is commonly used with FAB and EI ionization, however they are not widely used for electrospray and MALDI ionization sources primarily because of the much higher cost of these instruments. But in theory, they can be employed to practice the instant invention.

ESI and MALDI-MS commonly use quadrupole and time-of-flight mass analyzers, respectively. The limited resolution offered by time-of-flight mass analyzers, combined with adduct formation observed with MALDI-MS, results in accuracy on the order of 0.1% to a high of 0.01%, while ESI typically has an accuracy on the order of 0.01%. Both ESI and MALDI are now being coupled to higher resolution mass analyzers such as the ultrahigh resolution (>10⁵) mass analyzer. The result of increasing the resolving power of ESI and MALDI mass spectrometers is an increase in accuracy for biopolymer analysis.

Fourier-transform ion cyclotron resonance (FTMS) offers two distinct advantages, high resolution and the ability to tandem mass spectrometry experiments. FTMS is based on the principle of a charged particle orbiting in the presence of a magnetic field. While the ions are orbiting, a radio frequency (RF) signal is used to excite them and as a result of this RF excitation, the ions produce a detectable image current. The time-dependent image current can then be Fourier transformed to obtain the component frequencies of the different ions which correspond to their m/z.

Coupled to ESI and MALDI, FTMS offers high accuracy with errors as low as ±0.001%. The ability to distinguish individual isotopes of a protein of mass 29,000 is demonstrated.

A time-of-flight (TOF) analyzer is one of the simplest mass analyzing devices and is commonly used with MALDI ionization. Time-of-flight analysis is based on accelerating a set of ions to a detector with the same amount of energy. Because the ions have the same energy, yet a different mass, the ions reach the detector at different times. The smaller ions reach the detector first because of their greater velocity and the larger ions take longer, thus the analyzer is called time-of-flight because the mass is determine from the ions' time of arrival.

The arrival time of an ion at the detector is dependent upon the mass, charge, and kinetic energy of the ion. Since kinetic energy (KE) is equal to ½ mv² or velocity v=(2KE/m)^{1/2}, ions will travel a given distance, d, within a time, t, where t is dependent upon their m/z.

The magnetic double-focusing mass analyzer has two distinct parts, a magnetic sector and an electrostatic sector. The magnet serves to separate ions according to their mass-to-charge ratio since a moving charge passing through a magnetic field will experience a force, and travel in a circular motion with a radius of curvature depending upon the m/z of the ion. A magnetic analyzer separates ions according to their radii of curvature, and therefore only ions of a given m/z will be able to reach a point detector at any given magnetic field. A primary limitation of typical magnetic analyzers is their relatively low resolution. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument.

The new ionization techniques are relatively gentle and do not produce a significant amount of fragment ions, this is in contrast to electron ionization (EI) which produces many fragment ions. To generate more information on the molecular ions generated in the ESI and MALDI ionization sources, it has been necessary to apply techniques such as tandem mass spectrometry (MS/MS), to induce fragmentation. Tandem mass spectrometry (abbreviated MSn—where n refers to the number of generations of fragment ions being analyzed) allows one to induce fragmentation and mass analyze the fragment ions. This is accomplished by collisionally generating fragments from a particular ion and then mass analyzing the fragment ions.

Tandem mass spectrometry or post source decay is used for proteins that cannot be identified by peptide-mass matching or to confirm the identity of proteins that are tentatively identified by an error-tolerant peptide mass search, described above. This method combines two consecutive stages of mass analysis to detect secondary fragment ions that are formed from a particular precursor ion. The first stage serves to isolate a particular ion of a particular peptide (polypeptide) of interest based on its m/z. The second stage is used to analyze the product ions formed by spontaneous or induced fragmentation of the selected ion precursor. Interpretation of the resulting spectrum provides limited sequence information for the peptide of interest. However, it is faster to use the masses of the observed peptide fragment ions to search an appropriate protein sequence database and identify the protein as described in Griffin et al, Rapid Commun. Mass. Spectrom. 1995, 9: 1546. Peptide fragment ions are produced primarily by breakage of the amide bonds that join adjacent amino acids. The fragmentation of peptides in mass spectrometry has been well described (Falick et al., J. Am Soc. Mass Spectrom. 1993, 4, 882-893; Bieniann, K., Biomed. Environ. Mass Spectrom. 1988, 16, 99-111).

For example, fragmentation can be achieved by inducing ion/molecule collisions by a process known as collision-induced dissociation (CID) or also known as collision-activated dissociation (CAD). CID, is accomplished by selecting an ion of interest with a mass filter/analyzer and introducing that ion into a collision cell. A collision gas (typically Ar, although other noble gases can also be used) is introduced into the collision cell, where the selected ion collides with the argon atoms, resulting in fragmentation. The fragments can then be analyzed to obtain a fragment ion spectrum. The abbreviation MSn is applied to processes which analyze beyond the initial fragment ions (MS2) to second (MS3) and third generation fragment ions (MS4). Tandem mass analysis is primarily used to obtain structural information, such as protein or polypeptide sequence, in the instant invention.

In certain instruments, such as those by JEOL USA, Inc. (Peabody, Mass.), the magnetic and electric sectors in any JEOL magnetic sector mass spectrometer can be scanned together in “linked scans” that provide powerful MS/MS capabilities without requiring additional mass analyzers. Linked scans can be used to obtain product-ion mass spectra, precursor-ion mass spectra, and constant neutral-loss mass spectra. These can provide structural information and selectivity even in the presence of chemical interferences. Constant neutral loss spectrum essentially “lifts out” only the interested peaks away from all the background peaks, hence removing the need for class separation and purification. Neutral loss spectrum can be routinely generated by a number of commercial mass spectrometer instruments (such as the one used in the Example section). JEOL mass spectrometers can also perform fast linked scans for GC/MS/MS and LC/MS/MS experiments.

Once the ion passes through the mass analyzer it is then detected by the ion detector, the final element of the mass spectrometer. The detector allows a mass spectrometer to generate a signal (current) from incident ions, by generating secondary electrons, which are further amplified. Alternatively some detectors operate by inducing a current generated by a moving charge. Among the detectors described, the electron multiplier and scintillation counter are probably the most commonly used and convert the kinetic energy of incident ions into a cascade of secondary electrons. Ion detection can typically employ Faraday Cup, Electron Multiplier, Photomultiplier Conversion Dynode (Scintillation Counting or Daly Detector), High-Energy Dynode Detector (HED), Array Detector, or Charge (or Inductive) Detector.

The introduction of computers for MS work entirely altered the manner in which mass spectrometry was performed. Once computers were interfaced with mass spectrometers it was possible to rapidly perform and save analyses. The introduction of faster processors and larger storage capacities has helped launch a new era in mass spectrometry. Automation is now possible allowing for thousands of samples to be analyzed in a single day. Te use of computer also helps to develop mass spectra databases which can be used to store experimental results. Software packages not only helped to make the mass spectrometer more user friendly but also greatly expanded the instrument's capabilities.

The ability to analyze complex mixtures has made MALDI and ESI very useful for the examination of proteolytic digests, an application otherwise known as protein mass mapping. Through the application of sequence specific proteases, protein mass mapping allows for the identification of protein primary structure. Performing mass analysis on the resulting proteolytic fragments thus yields information on fragment masses with accuracy approaching ±15 ppm, or ±0.005 Da for a 1,000 Da peptide. The protease fragmentation pattern is then compared with the patterns predicted for all proteins within a database and matches are statistically evaluated. Since the occurrence of Arg and Lys residues in proteins is statistically high, trypsin cleavage (specific for Arg and Lys) generally produces a large number of fragments which in turn offer a reasonable probability for unambiguously identifying the target protein.

The primary tools in these protein identification experiments are mass spectrometry, proteases, and computer-facilitated data analysis. As a result of generating intact ions, the molecular weight information on the peptides/proteins are quite unambiguous. Sequence specific enzymes can then provide protein fragments that can be associated with proteins within a database by correlating observed and predicted fragment masses. The success of this strategy, however, relies on the existence of the protein sequence within the database. With the availability of the human genome sequence (which indirectly contain the sequence information of all the proteins in the human body) and genome sequences of other organisms (mouse, rat, Drosophila, C. elegans, bacteria, yeasts, etc.), identification of the proteins can be quickly determined simply by measuring the mass of proteolytic fragments.

Representative mass spectrometry instruments useful for practicing the instant invention are described in detail in the Examples. A skilled artisan should readily understand that other similar instruments with equivalent function/specification, either commercially available or user modified, are suitable for practicing the instant invention.

Protease Digestion

Prior to analysis by mass spectrometry, the protein may be chemically or enzymatically digested. For protein bands from gels, the protein sample in the gel slice may be subjected to in-gel digestion. (see Shevchenko A. et al., Mass Spectrometric Sequencing of Proteins from Silver Stained Polyacrylamide Gels. Analytical Chemistry 1996, 58: 850).

One aspect of the instant invention is that peptide fragments ending with lysine or arginine residues can be used for sequencing with tandem mass spectrometry. While trypsin is the preferred the protease, many different enzymes can be used to perform the digestion to generate peptide fragments ending with Lys or Arg residues. For instance, in page 886 of a 1979 publication of Enzymes (Dixon, M. et al. ed., 3rd edition, Academic Press, New York and San Francisco, the content of which is incorporated herein by reference), a host of enzymes are listed which all have preferential cleavage sites of either Arg- or Lys- or both, including Trypsin [EC 3.4.21.4], Thrombin [EC 3.4.21.5], Plasmin [EC 3.4.21.7], Kallikrein [EC 3.4.21.8], Acrosin [EC 3.4.21.10], and Coagulation factor Xa [EC 3.4.21.6]. Particularly, Acrosin is the Trypsin-like enzyme of spermatoza, and it is not inhibited by α1-antitrypsin. Plasmin is cited to have higher selectivity than Trypsin, while Thrombin is said to be even more selective. However, this list of enzymes are for illustration purpose only and is not intended to be limiting in any way. Other enzymes known to reliably and predictably perform digestions to generate the polypeptide fragments as described in the instant invention are also within the scope of the invention.

Sequence and Literature Databases and Database Search

The raw data of mass spectrometry will be compared to public, private or commercial databases to determine the identity of polypeptides.

BLAST search can be performed at the NCBI's (National Center for Biotechnology Information) BLAST website. According to the NCBI BLAST website, BLAST® (Basic Local Alignment Search Tool) is a set of similarity search programs designed to explore all of the available sequence databases regardless of whether the query is protein or DNA. The BLAST programs have been designed for speed, with a minimal sacrifice of sensitivity to distant sequence relationships. The scores assigned in a BLAST search have a well-defined statistical interpretation, making real matches easier to distinguish from random background hits. BLAST uses a heuristic algorithm which seeks local as opposed to global alignments and is therefore able to detect relationships among sequences which share only isolated regions of similarity (Altschul et al., 1990, J. Mol. Biol. 215: 403-10). The BLAST website also offer a “BLAST course,” which explains the basics of the BLAST algorithm, for a better understanding of BLAST.

For protein sequence search, several protein-protein BLAST can be used. Protein BLAST allows one to input protein sequences and compare these against other protein sequences.

“Standard protein-protein BLAST” takes protein sequences in FASTA format, GenBank Accession numbers or GI numbers and compares them against the NCBI protein databases (see below).

“PSI-BLAST” (Position Specific Iterated BLAST) uses an iterative search in which sequences found in one round of searching are used to build a score model for the next round of searching. Highly conserved positions receive high scores and weakly conserved positions receive scores near zero. The profile is used to perform a second (etc.) BLAST search and the results of each “iteration” used to refine the profile. This iterative searching strategy results in increased sensitivity.

“PHI-BLAST” (Pattern Hit Initiated BLAST) combines matching of regular expression pattern with a Position Specific iterative protein search. PHI-BLAST can locate other protein sequences which both contain the regular expression pattern and are homologous to a query protein sequence.

“Search for short, nearly exact sequences” is an option similar to the standard protein-protein BLAST with the parameters set automatically to optimize for searching with short sequences. A short query is more likely to occur by chance in the database. Therefore increasing the Expect value threshold, and also lowering the word size is often necessary before results can be returned. Low Complexity filtering has also been removed since this filters out larger percentage of a short sequence, resulting in little or no query sequence remaining. Also for short protein sequence searches the Matrix is changed to PAM-30 which is better suited to finding short regions of high similarity.

The databases that can be searched by the BLAST program is user selected, and is subject to frequent updates at NCBI. The most commonly used ones are:

Nr: All non-redundant GenBank CDS translations+PDB+SwissProt+PIR+PRF;

Month: All new or revised GenBank CDS translation+PDB+SwissProt+PIR+PRF released in the last 30 days;

Swissprot: Last major release of the SWISS-PROT protein sequence database (no updates);

Drosophila genome: Drosophila genome proteins provided by Celera and Berkeley Drosophila Genome Project (BDGP);

S. cerevisiae: Yeast (Saccharomyces cerevisiae) genomic CDS translations;

Ecoli: Escherichia coli genomic CDS translations;

Pdb: Sequences derived from the 3-dimensional structure from Brookhaven Protein Data Bank;

Alu: Translations of select Alu repeats from REPBASE, suitable for masking Alu repeats from query sequences. It is available by anonymous FTP from the NCBI website. See “Alu alert” by Claverie and Makalowski, Nature vol. 371, page 752 (1994).

Some of the BLAST databases, like SwissProt, PDB and Kabat are complied outside of NCBI. Other like ecoli, dbEST and month, are subsets of the NCBI databases. Other “virtual Databases” can be created using the “Limit by Entrez Query” option.

The Welcome Trust Sanger Institute offer the Ensembl software system which produces and maintains automatic annotation on eukaryotic genomes. All data and codes can be downloaded without constraints from the Sanger Centre website. The Centre also provides the Ensembl's International Protein Index databases which contain more than 90% of all known human protein sequences and additional prediction of about 10,000 proteins with supporting evidence. All these can be used for database search purposes.

In addition, many commercial databases are also available for search purposes. For example, Celera has sequenced the whole human genome and offers commercial access to its proprietary annotated sequence database (Discovery™ database).

Various softwares can be employed to search these databases. The probability search software Mascot (Matrix Science Ltd.). Mascot utilizes the Mowse search algorithm and scores the hits using a probabilistic measure (Perkins et al., 1999, Electrophoresis 20: 3551-3567, the entire contents are incorporated herein by reference). The Mascot score is a function of the database utilized, and the score can be used to assess the null hypothesis that a particular match occurred by chance. Specifically, a Mascot score of 46 implies that the chance of a random hit is less than 5%. However, the total score consists of the individual peptide scores, and occasionally, a high total score can derive from many poor hits. To exclude this possibility, only “high quality” hits—those with a total score >46 with at least a single peptide match with a score of 30 ranking number 1—are considered.

Other similar softwares can also be used according to manufacturer's suggestion.

PubMed, available via the NCBI Entrez retrieval system, was developed by the National Center for Biotechnology Information (NCBI) at the National Library of Medicine (NLM), located at the National Institutes of Health (NIH). The PubMed database was developed in conjunction with publishers of biomedical literature as a search tool for accessing literature citations and linking to full-text journal articles at web sites of participating publishers.

Publishers participating in PubMed electronically supply NLM with their citations prior to or at the time of publication. If the publisher has a web site that offers full-text of its journals, PubMed provides links to that site, as well as sites to other biological data, sequence centers, etc. User registration, a subscription fee, or some other type of fee may be required to access the full-text of articles in some journals.

In addition, PubMed provides a Batch Citation Matcher, which allows publishers (or other outside users) to match their citations to PubMed entries, using bibliographic information such as journal, volume, issue, page number, and year. This permits publishers easily to link from references in their published articles directly to entries in PubMed.

PubMed provides access to bibliographic information which includes MEDLINE as well as:

The out-of-scope citations (e.g., articles on plate tectonics or astrophysics) from certain MEDLINE journals, primarily general science and chemistry journals, for which the life sciences articles are indexed for MEDLINE.

Citations that precede the date that a journal was selected for MEDLINE indexing.

Some additional life science journals that submit full text to PubMed Central and receive a qualitative review by NLM.

PubMed also provides access and links to the integrated molecular biology databases included in NCBI's Entrez retrieval system. These databases contain DNA and protein sequences, 3-D protein structure data, population study data sets, and assemblies of complete genomes in an integrated system.

MEDLINE is the NLM's premier bibliographic database covering the fields of medicine, nursing, dentistry, veterinary medicine, the health care system, and the pre-clinical sciences. MEDLINE contains bibliographic citations and author abstracts from more than 4,300 biomedical journals published in the United States and 70 other countries. The file contains over 11 million citations dating back to the mid-1960's. Coverage is worldwide, but most records are from English-language sources or have English abstracts.

PubMed's in-process records provide basic citation information and abstracts before the citations are indexed with NLM's MeSH Terms and added to MEDLINE. New in process records are added to PubMed daily and display with the tag [PubMed—in process]. After MeSH terms, publication types, GenBank accession numbers, and other indexing data are added, the completed MEDLINE citations are added weekly to PubMed.

Citations received electronically from publishers appear in PubMed with the tag [PubMed—as supplied by publisher]. These citations are added to PubMed Tuesday through Saturday. Most of these progress to In Process, and later to MEDLINE status. Not all citations will be indexed for MEDLINE and are tagged, [PubMed—as supplied by publisher].

The Batch Citation Matcher allows users to match their own list of citations to PubMed entries, using bibliographic information such as journal, volume, issue, page number, and year. The Citation Matcher reports the corresponding PMID. This number can then be used to easily to link to PubMed. This service is frequently used by publishers or other database providers who wish to link from bibliographic references on their web sites directly to entries in PubMed.

Separation of Polypeptide Complexes

Polypeptide separation schemes can achieved based on differences in the molecular properties such as size, charge and solubility. Protocols based on these parameters include SDS-PAGE (SDS-PolyAcrylamide Gel Electrophoresis), size exclusion chromatography, ion exchange chromatography, differential precipitation and the like. SDS-PAGE is well-known in the art of biology, and will not be described here in detail. See Molecular Cloning A Laboratory Manual, 2nd Ed., ed. by Sambrook, Fritsch and Maniatis (Cold Spring Harbor Laboratory Press: 1989).

Size exclusion chromatography, otherwise known as gel filtration or gel permeation chromatography, relies on the penetration of macromolecules in a mobile phase into the pores of stationary phase particles. Differential penetration is a function of the hydrodynamic volume of the particles. Accordingly, under ideal conditions the larger molecules are excluded from the interior of the particles while the smaller molecules are accessible to this volume and the order of elution can be predicted by the size of the polypeptide because a linear relationship exists between elution volume and the log of the molecular weight. Size exclusion chromatographic supports based on cross-linked dextrans e.g. SEPHADEX.RTM., spherical agarose beads e.g. SEPHAROSE.RTM. (both commercially available from Pharmacia AB. Uppsala, Sweden), based on cross-linked polyacrylamides e.g. BIO-GEL.RTM. (commercially available from BioRad Laboratories, Richmond, Calif.) or based on ethylene glycol-methacrylate copolymer e.g. TOYOPEARL HW65S (commercially available from ToyoSoda Co., Tokyo, Japan) are useful in the practice of this invention.

Precipitation methods are predicated on the fact that in crude mixtures of polypeptides the solubilities of individual polypeptides are likely to vary widely. Although the solubility of a polypeptide in an aqueous medium depends on a variety of factors, for purposes of this discussion it can be said generally that a polypeptide will be soluble if its interaction with the solvent is stronger than its interaction with polypeptide molecules of the same or similar kind. Without wishing to be bound by any particular mechanistic theory describing precipitation phenomena, it is nonetheless believed that the interaction between a polypeptide and water molecules occur by hydrogen bonding with several types of charged groups, and electrostatically as dipoles with uncharged groups, and that precipitants such as salts of monovalent cations (e.g., ammonium sulfate) compete with polypeptides for water molecules, thus at high salt concentrations, the polypeptides become “dehydrated” reducing their interaction with the aqueous environment and increasing the aggregation with like or similar polypeptides resulting in precipitation from the medium.

Ion exchange chromatography involves the interaction of charged functional groups in the sample with ionic functional groups of opposite charge on an adsorbent surface. Two general types of interaction are known. Anionic exchange chromatography mediated by negatively charged amino acid side chains (e.g. aspartic acid and glutamic acid) interacting with positively charged surfaces and cationic exchange chromatography mediated by positively charged amino acid residues (e.g. lysine and arginine) interacting with negatively charged surfaces.

More recently affinity chromatography and hydrophobic interaction chromatography techniques have been developed to supplement the more traditional size exclusion and ion exchange chromatographic protocols. Affinity chromatography relies on the interaction of the polypeptide with an immobilized ligand. The ligand can be specific for the particular polypeptide of interest in which case the ligand is a substrate, substrate analog, inhibitor or antibody. Alternatively, the ligand may be able to react with a number of polypeptides. Such general ligands as adenosine monophosphate, adenosine diphosphate, nicotine adenine dinucleotide or certain dyes may be employed to recover a particular class of polypeptides. One of the least biospecific of the affinity chromatographic approaches is immobilized metal affinity chromatography (IMAC), also referred to as metal chelate chromatography. IMAC introduced by Porath et al.(Nature 258:598-99(1975) involves chelating a metal to a solid support and then forming a complex with electron donor amino acid residues on the surface of a polypeptide to be separated.

Hydrophobic interaction chromatography was first developed following the observation that polypeptides could be retained on affinity gels which comprised hydrocarbon spacer arms but lacked the affinity ligand. Although in this field the term hydrophobic chromatography is sometimes used, the term hydrophobic interaction chromatography (HIC) is preferred because it is the interaction between the solute and the gel that is hydrophobic not the chromatographic procedure. Hydrophobic interactions are strongest at high ionic strength, therefore, this form of separation is conveniently performed following salt precipitations or ion exchange procedures. Elution from HIC supports can be effected by alterations in solvent, pH, ionic strength, or by the addition of chaotropic agents or organic modifiers, such as ethylene glycol. A description of the general principles of hydrophobic interaction chromatography can be found in U.S. Pat. No. 3,917,527 and in U.S. Pat. No. 4,000,098. The application of HIC to the purification of specific polypeptides is exemplified by reference to the following disclosures: human growth hormone (U.S. Pat. No. 4,332,717), toxin conjugates (U.S. Pat. No. 4,771,128), antihemolytic factor (U.S. Pat. No. 4,743,680), tumor necrosis factor (U.S. Pat. No. 4,894,439), interleukin-2 (U.S. Pat. No. 4,908,434), human lymphotoxin (U.S. Pat. No. 4,920,196) and lysozyme species (Fausnaugh, J. L. and F. E. Regnier, J. Chromatog. 359:131-146 (1986)).

The principles of IMAC are generally appreciated. It is believed that adsorption is predicated on the formation of a metal coordination complex between a metal ion, immobilized by chelation on the adsorbent matrix, and accessible electron donor amino acids on the surface of the polypeptide to be bound. The metal-ion microenvironment including, but not limited to, the matrix, the spacer arm, if any, the chelating ligand, the metal ion, the properties of the surrounding liquid medium and the dissolved solute species can be manipulated by the skilled artisan to affect the desired fractionation.

Not wishing to be bound by any particular theory as to mechanism, it is further believed that the more important amino acid residues in terms of binding are histidine, tryptophan and probably cysteine. Since one or more of these residues are generally found in polypeptides, one might expect all polypeptides to bind to IMAC columns. However, the residues not only need to be present but also accessible (e.g., oriented on the surface of the polypeptide) for effective binding to occur. Other residues, for example poly-histidine tails added to the amino terminus or carboxyl terminus of polypeptides, can be engineered into the recombinant expression systems by following the protocols described in U.S. Pat. No. 4,569,794.

Phosphoproteins can be isolated using IMAC as described above. However, they can also be isolated by other means. Specifically, phosphoproteins with phosphorylated tyrosine residues can be isolated with phospho-tyrosine specific antibodies. Likewise, phospho-serine/threonine specific antibodies can be used to isolate phosphoproteins with phosphorylated serine/threonine residues. Many of these antibodies are available as affinity purified forms, either as monoclonal antibodies or antisera or mouse ascites fluid. For example, phospho-Tyrosine monoclonal antibody (P-Tyr-102) is a high-affinity IgGl phospho-tyrosine antibody clone that is produced and characterized by Cell Signaling Technology (Beverly, Mass.). As determined by ELISA, P-Tyr-102 (Cat. No. 9416) binds to a larger number of phospho-tyrosine containing peptides in a manner largely independent of the surrounding amino acid sequences, and also interacts with a broader range of phospho-tyrosine containing polypeptides as indicated by 2D-gel Western analysis. P-Tyr-102 is highly specific for phospho-Tyr in peptides/proteins, shows no cross-reactivity with the corresponding nonphosphorylated peptides and does not react with peptides containing phospho-Ser or phospho-Thr instead of phospho-Tyr. It is expected that P-Tyr-102 will react with peptides/proteins containing phospho-Tyr from all species.

Phospho-threonine antibodies are also available. For example, Cell Signaling Technology also offer an affinity-purified rabbit polyclonal phospho-threonine antibody (P-Thr-Polyclonal, Cat. No. 9381) which binds threonine-phosphorylated sites in a manner largely independent of the surrounding amino acid sequence. It recognizes a wide range of threonine-phosphorylated peptides in ELISA and a large number of threonine-phosphorylated polypeptides in 2D analysis. It is specific for peptides/proteins containing phospho-Thr and shows no cross-reactivity with corresponding nonphosphorylated sequences. Phospho-Threonine Antibody (P-Thr-Polyclonal) does not cross-react with sequences containing either phospho-Tyrosine or phospho-Serine. It is expected that this antibody will react with threonine-phosphorylated peptides/proteins regardless of species of origin. Upstate Biotechnology (Lake Placid, N.Y.) also provides an anti-phospho-serine/threonine antibody with broad immunoreactivity for polypeptides containing phosphorylated serine and phosphorylated threonine residues.

Many other similar products are also available on the market. These antibodies can be readily coupled to supporting matrix materials to generate affinity columns according to standard molecular biology protocols (for details and general means of antibody production, see Using Antibodies: A Laboratory Manual: Portable Protocol NO. I, Harlow and Lane, Cold Spring Harbor Laboratory Press: 1998; also see Antibodies: A Laboratory Manual, edited by Harlow and Lane, Cold Spring Harbor Laboratory Press: 1988).

A similar approach can be applied towards the isolation of any specific polypeptide, against which specific antibodies are available.

The methods of the invention has been applied to secreted proteins from cultured cells undergoing adipogenesis. However, it should be understood that other samples with polypeptides from other sources, such as lysates of cell cultures or tissue samples can also be used.

The cell populations to be compared can be each independently from different tissue sources, developmental stages, and/or differentiation stages, such that expression levels of certain proteins at different conditions can be studied. The expression levels of proteins in different samples can be directly compared after normalization according to well-known procedures. For example, the expression of a specific protein in a sample can be expressed a percentage of a protein whose expression level rarely changes (such as actin, etc., depending on cell types). The expression levels can be quantitated or semi-quantitated, based on gel staining results on SDS-PAGE or data output in mass spectrometry.

The identification of agents whose expression level is substantially changed among different cell samples indicates that such agents may play an important role in adipogenesis. Thus it is possible to screen among these agents for candidate genes whose expression is sufficient to promote or inhibit adipogenesis. Such candiadte genes typically manifest their effects through affecting cells in culture in terms of proliferation, differentiation, survival, or expression of adipogenesis marker genes, wherein an adipogenic agonist increases or potentiates the growth, proliferation, differentiation, survival of cultured cells, or expression of certain differentiation marker genes, whereas an adipogenic antagonist decreases or inhibits the growth, proliferation, differentiation, survival of said cells, or inhibit the expression of certain differentiation marker genes.

In this respect, in addition to the identification of factors that had been previously recognized to participate in adipogenesis, the present invention also identified several proteins whose role in adipogenesis had not been appreciated. These factors provide the basis for novel therapeutics to modulate the growth, proliferation, differentiation, and survival of adipose tissue in order to treat conditions characterized by too much or insufficient adipogenic tissue. A detailed description of a few of these identified genes are described below.

Any of the identified agents may also be formulated with a pharmaceutically acceptable carrier or excipient for in vivo administration to a mammalian patient.

The instant invention also provides an effective way of identifying marker genes involved in adipogenesis. Such information may be used to establish a time-table of adipogenesis, and the specific genes associated with each specific differentiation stages, thereby determining exactly what differentiation stage a candidate cell might be at based on its expression profiles of those marker genes.

The following provides detailed description of a few genes identified using the methods of the instant invention.

Pigment Epithelium-Derived Factor (PEDF)

PEDF is a glycoprotein of approximately 50 kD previously identified as a factor secreted by fetal retinal pigment epithelial (RPE) cells (Steele et al., 1992). PEDF is a member of the serine protease inhibitor (serpin) superfamily, however the functional significance of this structural classification is unclear as the functions of PEDF on neuronal cell types can be recapitulated using truncated forms of the protein which lack the serpin domain.

PEDF has potent neurogenic properties, and promotes robust survival and neuronal differentiation in human Y79 retinoblastoma cells, as well as in motor neurons (Steele et al., 1992, Houenou et al., 1999). Additionally, PEDF has been shown to protect neuronal cells from damage and death following either hydrogen-peroxide induced or ischemic injury (Cao et al., 1999; Ogata et al., 2001). One potential mechanism whereby PEDF promotes neuronal survival following ischemic injury is by inhibiting angiogenesis. Such anti-angiogenic properties might function endogenously to prevent neovascularization in the eye; a process responsible for most cases of vision loss in the developed world (Dawson et al., 1999; Chader, 2001; Stellmach et al., 2001). As such, PEDF may represent an important treatment option for pathologies such as retinopathy and macular degeneration.

Despite the importance of PEDF in neuronal differentiation and survival, its function in adipogenesis had not been previously described. In the present invention, we report the identification of PEDF as a secreted factor differentially expressed between preadipocytes and adipocytes. The use of PEDF to influence adipogenesis in a cell is disclosed. In a preferred embodiment, an effective amount of PEDF is administered to a patient to modulate the growth, proliferation, differentiation, or survival of adipocytes. In another preferred embodiment, an expression cassette comprising a nucleic acid sequence encoding PEDF under the control of transcriptional initiation and termination regions is contemplated. In another preferred embodiment, a cell comprising an expression cassette encoding PEDF is contemplated. In another preferred embodiment, an effective amount of cells expressing PEDF are administered to a patient to modulate the growth, proliferation, differentiation, or survival of adipocytes. In another embodiment, PEDF is used in combination with other factors with additive, and possibly synergistic, affects on survival, growth, and differentiation.

Haptoglobin

Haptoglobin is an acute phase protein composed of a dimer of an α and β subunit that are derived from the processing of a single polypeptide chain, and is the principle hemoglobin binding protein (Hanley et al., 1983). Haptoglobin has been studied in hepatocytes, and its expression levels appear to respond to a variety of stimuli including pregnancy, infection, inflammation, trauma, and malignancy. Additional studies have demonstrated that haptoglobin is regulated by a variety of cytokines including IL-1, IL-6, and TGFβ, and by several drugs including forskolin and dexamethasone (Marinkovic et al., 1990; Friedrichs et al., 1995; Yu et al., 1999).

Despite the importance of haptoglobin in a variety of processes, its function in adipogenesis had not been previously described. In the present invention, we report the identification of haptoglobin as a secreted factor differentially expressed between preadipocytes and adipocytes. The use of haptoglobin to influence adipogenesis in a cell is disclosed. In a preferred embodiment, an effective amount of haptoglobin is administered to a patient to modulate the growth, proliferation, differentiation, or survival of adipocytes. In another preferred embodiment, an expression cassette comprising a nucleic acid sequence encoding haptoglobin under the control of transcriptional initiation and termination regions is contemplated. In another preferred embodiment, a cell comprising an expression cassette encoding haptoglobin is contemplated. In another preferred embodiment, an effective amount of cells expressing haptoglobin are administered to a patient to modulate the growth, proliferation, differentiation, or survival of adipocytes. In another embodiment, haptoglobin is used in combination with other factors with additive, and possibly synergistic, affects on survival, growth, and differentiation.

Hippocampal Cholinegic Neurostimulating Peptide (HCNP)

HCNP was originally identified in hippocampal tissue, and shown to cooperate with NGF during the development of medial septal nuclei (Ojika et al., 1992; Ojika et al., 1994). Analysis of HCNP expression in rat demonstrated that HCNP is expressed in many regions of the brain including the basal forebrain cholinergic system, the olfactory system, the cerebellum, the pyramidal cells of the CA3 region, the septal area, the piriform cortex, the entorhinal cortex, the thalamic nuclei, the subthalamic nuclei, the medial habenular nuclei, the substantia nigra, Purkinje cells of the cerebellum, and the choroid plexus. However, HCNP is not expressed in glial cells (Taiji et al., 1996).

Expression of HCNP protein is sensitive to NMDA receptor activation, and interestingly, increased levels of HCNP have been detected in the cerebrospinal fluid of some patients with Alzheimer's disease (Tsugu et al., 1998; Ojika et al., 1999).

Despite the importance of HCNP in many aspects of neuronal development, its function in adipogenesis had not been previously described. In the present invention, we report the identification of HCNP as a secreted factor differentially expressed between preadipocytes and adipocytes. The use of HCNP to influence adipogenesis in a cell is disclosed. In a preferred embodiment, an effective amount of HCNP is administered to a patient to modulate the growth, proliferation, differentiation, or survival of adipocytes. In another preferred embodiment, an expression cassette comprising a nucleic acid sequence encoding HCNP under the control of transcriptional initiation and termination regions is contemplated. In another preferred embodiment, a cell comprising an expression cassette encoding HCNP is contemplated. In another preferred embodiment, an effective amount of cells expressing HCNP are administered to a patient to modulate the growth, proliferation, differentiation, or survival of adipocytes. In another embodiment, HCNP is used in combination with other factors with additive, and possibly synergistic, affects on survival, growth, and differentiation.

Neutrophil Gelatinase Cholinergic Neurostimulating Peptide (NGAL)

NGAL is a 25 kD lipocalin originally purified from human neutrophils (Flowers et al., 1984). It exists in monomeric, homodimeric, and heterodimeric forms. Synthesis of NGAL is induced in cells during inflammation, and more recently NGAL has been identified as one of a number of genes up-regulated in inflammatory bowel disease (Kjeldsen et al., 2000; Lawrence et al., 2001)

Despite the importance of NGAL in the inflammatory response, its function in adipogenesis had not been previously described. In the present invention, we report the identification of NGAL as a secreted factor differentially expressed between preadipocytes and adipocytes. The use of NGAL to influence adipogenesis in a cell is disclosed. In a preferred embodiment, an effective amount of NGAL is administered to a patient to modulate the growth, proliferation, differentiation, or survival of adipocytes. In another preferred embodiment, an expression cassette comprising a nucleic acid sequence encoding NGAL under the control of transcriptional initiation and termination regions is contemplated. In another preferred embodiment, a cell comprising an expression cassette encoding NGAL is contemplated. In another preferred embodiment, an effective amount of cells expressing NGAL are administered to a patient to modulate the growth, proliferation, differentiation, or survival of adipocytes. In another embodiment, NGAL is used in combination with other factors with additive, and possibly synergistic, affects on survival, growth, and differentiation.

Stromal Cell Derived Factor-1 (SDF-1)

SDF-1 is a CXC chemokine, and a ligand for CXCR4/fusin. The significance of SDF-1 in the immune system has long been appreciated. This factor stimulates proliferation of B-cells, and synergistically augments the ability of IL-7 to stimulate B-cell proliferation (Tashiro et al., 1993; Nagasawa et al., 1994; Oberlin et al., 1996; Bleul et al., 1996). More recently, an anti-infection activity of SDF-1 has been identified. Cell transfected with SDF-1 appear to be resistant to infection by lymphocytic HIV strains (Oberlin et al., 1996; Bleul et al., 1996).

The identification of a factor known for its roles in proliferation and cell survival in the immune system in adipocytes suggests that SDF-1 may help to regulate cell proliferation or behavior is adipose tissue as well. The invention contemplates the use of SDF-1 to influence adipogenesis in a cell. In a preferred embodiment, an effective amount of SDF-1 is administered to a patient to modulate the growth, proliferation, differentiation, or survival of adipocytes. In another preferred embodiment, an expression cassette comprising a nucleic acid sequence encoding SDF-1 under the control of transcriptional initiation and termination regions is contemplated. In another preferred embodiment, a cell comprising an expression cassette encoding SDF-1 is contemplated. In another preferred embodiment, an effective amount of cells expressing SDF-1 are administered to a patient to modulate the growth, proliferation, differentiation, or survival of adipocytes. In another embodiment, SDF-1 is used in combination with other factors with additive, and possibly synergistic, affects on survival, growth, and differentiation.

Calumenin and Calvasculin

Calumenin and calvasculin are two calcium binding proteins expressed by adipocytes (Jackson-Grusby et al., 1987; Yabe et al., 1997). The expression of two calcium binding proteins in adipocytes suggests that utilization and responsiveness to calcium may be an important mechanism of regulation in adipose tissue, and the identification of such proteins offers novel methods to modulate the growth, proliferation, differentiation, or survival of adipocytes.

The invention contemplates the use of calumenin or calvasculin to influence adipogenesis in a cell. In a preferred embodiment, an effective amount of calumenin or calvasculin is administered to a patient to modulate the growth, proliferation, differentiation, or survival of adipocytes. In another preferred embodiment, an expression cassette comprising a nucleic acid sequence encoding calumenin or calvasculin under the control of transcriptional initiation and termination regions is contemplated. In another preferred embodiment, a cell comprising an expression cassette encoding calumenin or calvasculin is contemplated. In another preferred embodiment, an effective amount of cells expressing calumenin or calvasculin are administered to a patient to modulate the growth, proliferation, differentiation, or survival of adipocytes. In another embodiment, calumenin or calvasculin is used in combination with other factors with additive, and possibly synergistic, affects on survival, growth, and differentiation.

Colligen-1

Colligen-1 is a protease inhibitor whose role in adipogenesis had not been recognized (Abrahamson et al., 1987; Clarke and Sanwal, 1992). Interestingly, it is not the only protease inhibitor isolated by the invention which also identified cystatin-C as a protease inhibitor secreted by adipocytes. Cystatin-C had been previously identified in a cDNA based screen (Tsuruga et al., 2000). Thus, our results confirm that cystatin-C is regulated during adipogenesis at both the RNA and protein levels, and also identify a second novel protease inhibitor expressed during adipogenesis: colligen-1.

The expression of two protease inhibitors in differentiated adipocytes underscores their likely importance during adipogenesis. The invention contemplates the use of colligen-1 to influence adipogenesis in a cell. In a preferred embodiment, an effective amount of colligen-1 is administered to a patient to modulate the growth, proliferation, differentiation, or survival of adipocytes. In another preferred embodiment, an expression cassette comprising a nucleic acid sequence encoding colligen-1 under the control of transcriptional initiation and termination regions is contemplated. In another preferred embodiment, a cell comprising an expression cassette encoding colligen-1 is contemplated. In another preferred embodiment, an effective amount of cells expressing colligen-1 are administered to a patient to modulate the growth, proliferation, differentiation, or survival of adipocytes. In another embodiment, colligen-1 is used in combination with other factors with additive, and possibly synergistic, affects on survival, growth, and differentiation.

Gelsolin

Gelsolin is an actin binding protein found in plasma and other tissues (Kwiatkowski et al., 1986). Given the extensive cellular remodeling that accompanies adipogenesis, it is not surprising that many extracellular matrix related proteins are differentially expressed during adipogenesis. Many such molecules were identified using the approaches describes herein including fibronectin, type I collagen α2, and type VI collagen α3. Manipulation of extra-cellular matrix molecules, with there concomitant effects on cell shape, may present a novel means for regulating the growth, proliferation, differentiation, and survival of adipocytes.

The invention contemplates the use of gelsolin to influence adipogenesis in a cell. In a preferred embodiment, an effective amount of gelsolin is administered to a patient to modulate the growth, proliferation, differentiation, or survival of adipocytes. In another preferred embodiment, an expression cassette comprising a nucleic acid sequence encoding gelsolin under the control of transcriptional initiation and termination regions is contemplated. In another preferred embodiment, a cell comprising an expression cassette encoding gelsolin is contemplated. In another preferred embodiment, an effective amount of cells expressing gelsolin are administered to a patient to modulate the growth, proliferation, differentiation, or survival of adipocytes. In another embodiment, gelsolin is used in combination with other factors with additive, and possibly synergistic, affects on survival, growth, and differentiation.

Osteoblast Specific Factor 2

Osteoblast specific factor 2 (fasciclin I-like) is a protein highly expressed in bone and lung tissues that functions as an adhesion molecule in bone formation (Takeshita et al., 1993). The human homologue periostin has been demonstrated to be secreted and upregulated in epithelial ovarian tumors, and to serve as diagnostic marker for cell lung carcinomas (Sasaki et al., 2001, Gillan et al., 2002). In a preferred embodiment an effective amount of osteoblast specific factor is administered to a patient to regulate the development of adipose tissue. In another preferred embodiment osteoblast specific factor is used in combination with other factors to influence the adipose conversion in tissues where, due to aging or disease, there is an accelerated formation, but not maturation, of adipocytes.

Follistatin-Like Protein

Follistatin-like protein binds and neutralizes both activin, a member of the transforming growth factor-beta, and bone morphogenic protein-2, thereby regulating the signal transduction pathways induced by these factors (Tsuchida et al., 2000). In a preferred embodiment an effective amount of osteoblast specific factor is administered to patient to regulate the development of adipose tissue by inhibiting the growth factor induced cascade. In another preferred embodiment follistatin-like protein is used in combination with other factors to influence the processes of proliferation and differentiation.

Calgizzarin

Calgizzarin belongs to the S-100 family and contains two calcium-binding domains. It is a cytokine that activates the host immune-response mechanisms by activating endothelial monocytes. In addition it has been found to be up-regulated in breast and colon cancer. In a preferred embodiment an effective amount of calgizzarin is administered to a patient to regulate calcium-mediated signaling in adipose tissue. In another preferred embodiment calgizzarin is used in combination with other factors to influence the development of adipocytes.

### EXAMPLES

The invention now being generally described, it will be more readily understood by reference to the following examples, which are included merely for purposes of illustration of certain aspects and embodiments of the present invention, and are not intended to limit the invention.

### Example 1

Pilot Screen Demonstrates that the Differential Expression of Polypeptides is Detectable Over Time

The present invention aims to identify and characterize proteins differentially expressed during adipogenesis. We undertook a pilot screen to demonstrate that differential protein expression was detectable, and also to determine the optimal time points during the differentiation protocol to make comparisons of protein expression.

3T3-L1 preadipocytes are a very useful system for studying adipogenesis. This preadipocyte cell line differentiates into adipocytes when cultured under certain conditions. 3T3-L1 preadipocytes were grown at 37° C. in 10% CO₂ in DMEM plus 10% fetal bovine serum supplemented with antibiotics. Cells were grown to confluence for two days. The confluent, uninduced cells are considered day 0 preadipocytes. The cells are induced to differentiate by changing the culture medium to DMEM containing 10% fetal bovine serum, 0.5 mM 3-isobutyl-1-methyxanthine (Sigma), 1 μm dexamethasone (Sigma), and 167 nM insulin (Novo Nordisk). After 48 hours (day 2), the medium is replaced with DMEM plus 10% fetal bovine serum and 167 nM insulin. After another 48 hours (day 4), the medium is replaced and the cells are now cultured in DMEM plus 10% fetal bovine serum. This medium is replaced every 48 hours for the remainder of the culture period.

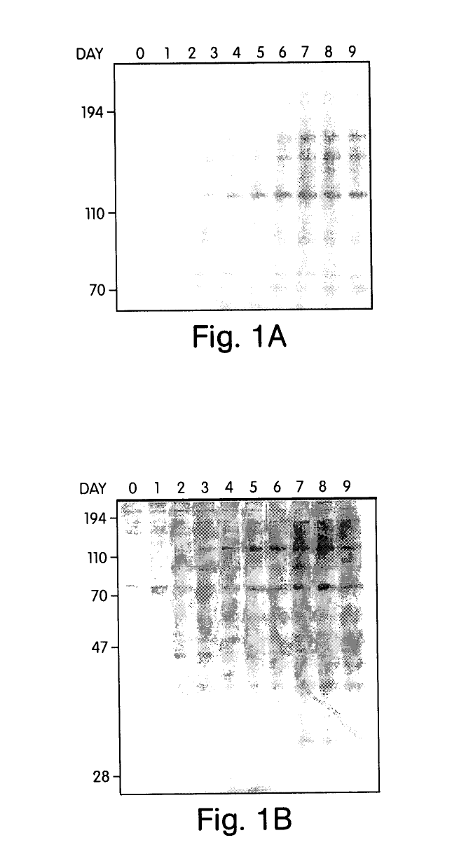

FIGS. 1A and B show a time course of ³⁵S-labeled proteins secreted from preadipocytes and adipocytes over several days of exposure to differentiation conditions. For metabolic labeling of proteins, the cells were grown as described above. The cells were washed with serum free media and labeled with ³⁵S-labeled methionine for 6 hours at every time point in the differentiation protocol. Radiolabeled supernatants were harvested, separated by SDS-PAGE on either a 7% (1A) or 15% (1B) acrylamide gel. The radiolabeled polypeptides were visualized by autoradiography.

This time course demonstrates that the detection of differentially expressed polypeptides is feasible. Several proteins in both the high and the low molecular weight range are observable and differentially expressed by three or four days of the differentiation protocol, and by nine days of the differentiation protocol there is robust secretion of many proteins in both the high and the low molecular weight range.

This pilot study demonstrates the feasibility of using this method to identify differentially expressed proteins between preadipocytes and adipocytes. For further analysis, we compared proteins secreted by either day 0 preadipocytes or day 9 adipocytes.

### Example 2

Proteomic Approach Identifies Known Proteins

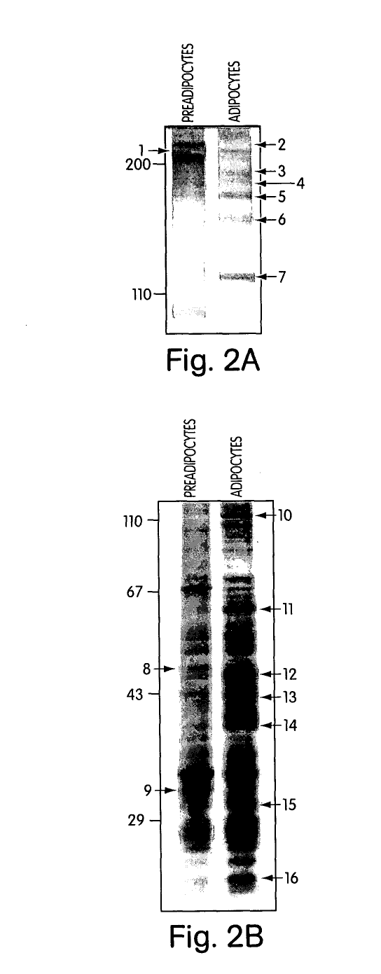

Cells were cultured as described above, and the supernatants from day 0 preadipocytes and day 9 adipocytes were harvested, separated by SDS-PAGE, and visualized by silver staining (FIGS. 2A, B). Examination of these gels revealed a number of major bands differentially expressed in either the preadipocyte or the adipocyte cell population. Several of these differentially expressed bands were analyzed by nanospray mass spectrometry.

Bands were excised from one-dimensional, silver stained gels. The bands were alkylated and reduced using previously described methods (Shevchenko et al., 1996; Wilm et al., 1996). The bands were then in-gel digested with an excess-of trypsin overnight at 37° C. (Promega). This supernatant was acidified via treatment with formic acid and loaded onto a Poros R2™ micro-column (Perspective Biosystems) and desalted by previously described methods (Gobom et al., 1999). The peptides were eluted with methanol/5% formic acid directly into a nanoelectrospray needle (MDS-Proteomics). Nanospray tandem mass spectrometry analysis was performed either on Q-TOF mass spectrometer (Micromass) or on QSTAR Pulsar (PE Sciex) equipped with a nanoelectrospray source (MDS-Proteomics). From this analysis, fragmentation spectra were obtained, and the resulting peptide sequence tags were used to search the nrdb database (EBI) using the PepSea program (Protana).

Using this approach, we have identified several proteins that had been previously identified as being involved in adipogenesis. Two of these previously identified proteins are down regulated during adipogenesis: fibronectin and procollagen type I α2 (Weiner et al., 1989; Zhou et al., 1999; Slevarajan et al., 2001). Differential expression of matrix proteins such as these is likely critical for the extensive cell remodeling that accompanies adipogenesis.

Our analysis also identified several up-regulated secreted proteins. Adipocyte complement-related protein 30 kDa (Acrp 30), complement factor C3 precursor and adipsin were found to be mainly produced by adipocytes. Acrp 30 is a protein known to be secreted exclusively by adipocytes and its mRNA is induced 100-fold during the process of adipocyte differentiation (Scherer et al., 1995). It was also cloned in an independent study and designated as adipoQ (Hu et al., 1996). Acrp 30 has four domains—its C-terminal globular domain was recently shown to increase fatty acid oxidation in muscle and to cause weight loss in mice when they were put on a regimen of high fat and high sucrose diet (Fruebis et al., 2001). Acrp 30 presumably undergoes proteolytic cleavage in vivo to produce a C-terminal fragment containing the globular domain alone which migrates at 16 kD (Fruebis et al., 2001). In this study, we have identified Acrp 30 from a band that migrates at 30 kD indicating that it is the uncleaved version of Acrp 30.

Complement factor C3 was identified from bands 5, 7, 10 and 11. Its mRNA and protein levels have previously been shown to increase dramatically as preadipocytes differentiate into adipocytes (Choy et al., 1992; Cianflone et al., 1994). Activation of C3 is a central step in the alternative complement pathway. The complement factor C3 precursor, which is approximately 200 kDa is composed of alpha and beta chains that are linked by a disulfide bond (Esterbauer et al., 1999). The form of C3 migrating at 110 kDa that we have identified is the alpha chain whereas the form migrating at 70 kDa is the beta chain. C3a and C3b are derived by proteolytic cleavage of the complement C3 precursor and correspond to its N and C-terminus, respectively. Cleavage of C3a to C3adesArg makes it capable of inducing triglyceride synthesis and glucose transport indicating its intimate involvement in energy metabolism adipocytes (Baldo et al., 1993; Maslowska et al., 1997; Murray et al., 1997).

Adipsin was identified from bands 12 and 14 as an up-regulated protein. It was originally isolated as an mRNA species that was up-regulated over 200-fold during adipocyte conversion process (Spiegelman et al., 1983). It was subsequently also shown to be up-regulated at the protein level (Kitagawa et al., 1989) and is secreted in two forms that differ in their glycosylation patterns—37 kD and 44 kD (Cook et al., 1987)—both of these alternative forms of adipsin were identified in our study.

Entactin/nidogen was another protein that we identified as an up-regulated protein. It was identified by Tsuruga and colleagues as a differentially expressed mRNA using a signal sequence trap method (Tsuruga et al., 2000) and was shown to be up-regulated 30-fold at the protein level during adipocyte differentiation using immunoprecipitating antibodies (Aratani et al., 1988). Entactin can form a ternary complex with type IV collagen and laminin thereby helping in the formation of the basement membrane (Aratani et al., 1988).

We found collagen type VI alpha 3 to be secreted mainly by adipocytes confirming the results of a recent study that found this collagen expressed mainly in adipocytes using a cDNA based subtraction strategy (Imagawa et al., 1999). We had also identified the alpha 3 subunit of type VI collagen as a protein up-regulated in adipogenesis by our subtractive antibody screening method (Scherer et al, 1998).

Table 1 summarizes the differentially expressed secreted proteins identified by this proteomic approach. The band numbers provided correspond to the numbered arrows in FIG. 2. The identification of proteins previously shown to be involved in adipogenesis demonstrates the effectiveness of this approach.

### Example 3

Identification of Novel Proteins

In addition to the previously identified proteins described above, the method summarized in Example 2 also identified several proteins that had never been implicated in adipogenesis (Summarized in Table 1). These proteins have been studied in other developmental contexts, but their role in adipogenesis has gone unappreciated until now. The identification of novel proteins involved in adipogenesis demonstrates the power of this proteomics based approach to provide new insights into the study of adipogenesis, and offers novel therapies for conditions characterized by hyper or hypo-adiopogenesis.

(a) Pigment Epithelium Derived Factor (PEDF)

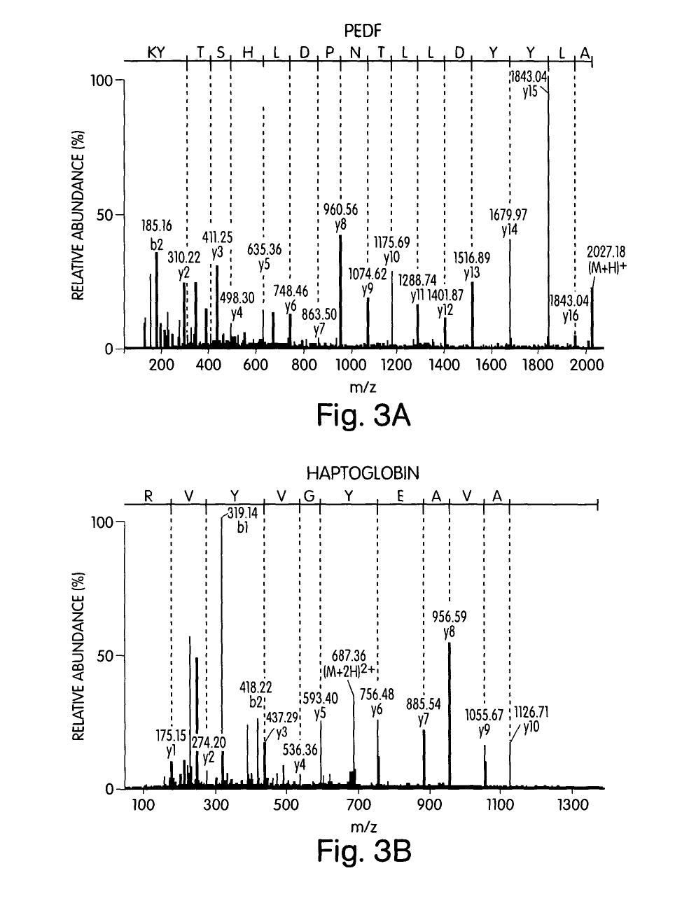

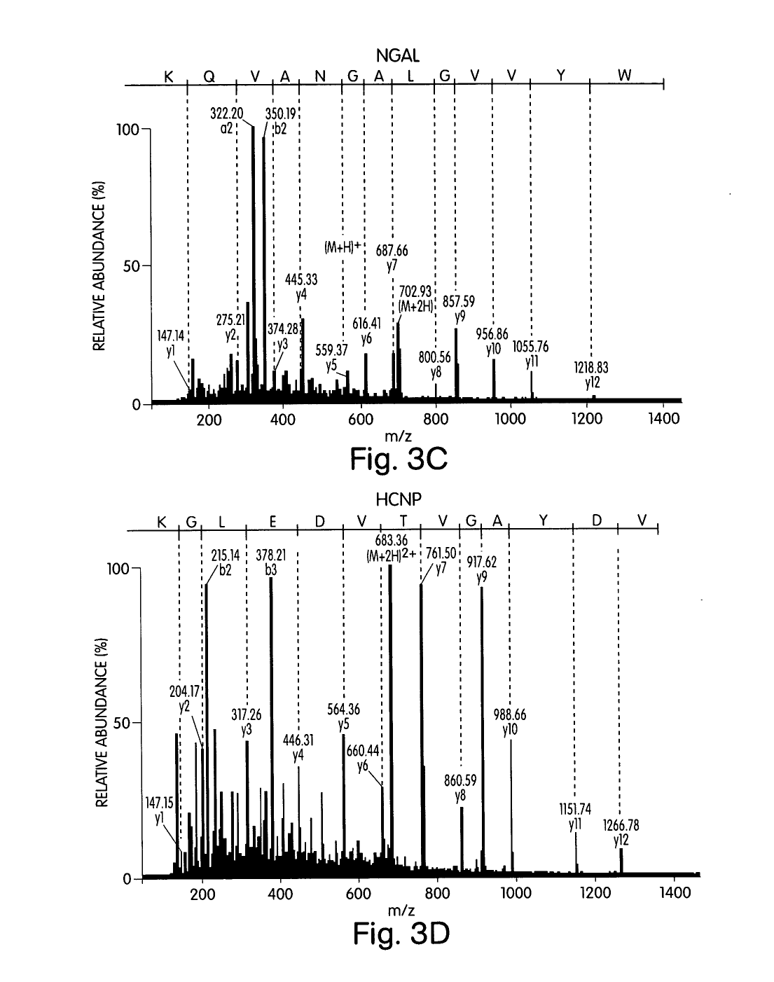

Band 8 (see FIG. 2B) corresponds to a factor of about 50 kD secreted from preadipocytes but not from adipocytes. Mass spectrometry analysis revealed that this 50 kD factor is pigment epithelium derived factor (PEDF) (FIG. 3A). PEDF belongs to the serine protease inhibitor, or serpin, family, and has been shown to play a role in retina development (Tombran-Tink et al., 1991; Shirozu et al., 1996). Despite several studies demonstrating that PEDF can induce differentiation in neuronal cell cultures, can have neuroprotective effects in mouse models of retinopathy, and can inhibit angiogenesis, there has been no suggestion that PEDF has a function during adipogenesis (Steele et al., 1993; Dawson et al., 1999; Stellmach et al., 2001). The identification of this factor, previously shown to have potent affects in vivo on cell fate and differentiation, during adipogenesis provides another therapeutic target for the treatment of adipogenic conditions.

(b) Haptoglobin

Several of the bands corresponding to proteins up-regulated in adipocytes were found to be haptoglobin (bands 12, 13, 14—see FIG. 3B and Table 1). The invention identified partially and fully glycosylated forms of prohaptoglobin which migrate at approximately 45 kD and 48 kD, as well as the core glycosylated β subunit migrating at approximately 38 kD.

Haptoglobin is an acute phase protein synthesized by the liver, and is the principle hemoglobin binding protein (Hanley et al., 1983). Haptoglobin has been studied in hepatocytes, and its expression levels appear to respond to a variety of stimuli including pregnancy, infection, inflammation, trauma, and malignancy. Additional studies have demonstrated that haptoglobin is regulated by a variety of cytokines including IL-1, IL-6, and TGFβ, and by several drugs including forskolin and dexamethasone (Marinkovic et al., 1990; Friedrichs et al., 1995; Yu et al., 1999).

The identification of this factor, previously shown to be involved in the cellular response to many processes including inflammation, trauma, infection, and malignancy, during adipogenesis provides another therapeutic target for the treatment of adipogenic conditions.

(c) Neutrophil Gelatinase Associated Lipocalin (NGAL)

NGAL corresponds to band 16, and is expressed preferentially in adipocytes (FIG. 3C). NGAL belongs to the family of fatty acid binding proteins called lipocalins (Flower et al., 1991), and was originally characterized as an oncogene induced upon infection of cells with either polyoma or SV 40 virus (Hraba-Renevey et al., 1989; Bundgaard et al., 1994). More recently, induction of NGAL has been recognized as a more general response to inflammation, and it appears to be one of many genes up-regulated in inflammatory bowel disease (Kjeldsen et al., 2000; Lawrence et al., 2001)

The identification of this factor, previously shown to have affects on cellular proliferation, differentiation, and survival, during adipogenesis provides another therapeutic target for the treatment of adipogenic conditions.

(d) Hippocampal Cholinergic Neurostimulating Peptide (HCNP)

HCNP was also identified from band 16, and is expressed preferentially in adipocytes (FIG. 3D). HCNP had been previously identified in hippocampal tissues where it functions cooperatively with NGF in the development of medial septal nuclei (Ojika et al., 1992; Ojika et al., 1994).

The identification of this factor, previously shown to be involved in proper neuronal development, during adipogenesis provides another therapeutic target for the treatment of adipogenic conditions.

### Example 4

mRNA Analysis of Factors Differentially Expressed During Adipogenesis

The differentially expressed factors described herein were identified based on differential protein expression. However, this method does not distinguish between factors that are regulated post-transcriptionally, and factors that are regulated transcriptionally. Therefore, it seemed likely that some of these factors are also differentially expressed at the level of the mRNA (transcriptionally regulated) while other factors are only differentially expressed at the level of the protein (post-transcriptionally regulated).

mRNA expression in preadipocytes and day 9 adipocytes was examined by RT-PCR and Northern blot analysis.

(a) RT-PCR analysis: Total RNA was prepared from preadipocytes and from day 9 adipocytes. Reverse transcription reactions were performed in a volume of 25 μl containing 1 μg of total RNA, 3 μg of random hexamers (Amersham Pharmacia Biotech), 50 mM Tris-HCl, pH 8.3, 75 mM KCl, 3 mM MgCl₂, 10 mM dithiothreitrol, 40 units of RNA-guard (Amersham Pharmacia Biotech), 0.9 mM dNTPs, and 200 units of Moloney murine leukemia virus reverse transcriptase (Life Technologies). Reactions were left for 10 minutes at room temperature, followed by incubation at 37° C. for 1 hour. After cDNA synthesis, the reaction mix was diluted with 50 μl of water. Multiplex RT-PCR was performed by the methods previously described (Hansen et al., 1999). Briefly, the PCR reaction was performed in 25 μl volume containing 1.5 μl of diluted cDNA, 50 mM KCl, 10 mM Tris-HCl, pH 9.0, 1.5 mM MgCl₂, 0.1% Triton X-100, 40 μM dATP, dTTP and dGTP, 20 μM dCTP, 5 pmol of each primer, 1.25 units of Taq polymerase and 1.25 μCi of α-³²P dCTP (6000 Ci/mmol) (NEN Life Science Products). The reaction mix was denatured by heating at 94° C. for 1 min. Denaturation was followed by 15, 20 or 25 cycles (depending on the set of primers used) of 94° C. for 30 seconds, 55° C. for 60 seconds, and 72° C. for 40 seconds. All reactions contained the TATA-binding protein (TBP) primer set as an internal standard. Reactions amplifying NGAL were performed with 25 cycles, adipsin and ACRP 30 with 15 cycles and PEDF, HCNP and haptoglobin with 20 cycles. Ten microliters of each reaction were dried down and resuspended in formamide dye mix (98% deionized formamide, 10 mM EDTA, pH 8.0, 0.2% bromophenol blue, 0.2% xylene cyanol) and loaded onto 0.4 mm, 8 M urea, 1×TBE, 6% polyacrylamide gels. Electrophoresis was performed for 3 hours at 50 watts. The gels were dried and exposed overnight on a PhosphorImage storage screen and subsequently scanned on a PhosphorImage plate (Molecular Dynamics).

The following primer pairs were used for multiplex RT-PCR:

| | | |
|---|---|---|
| PEDF: | (upstream)-GCGAACTTACCAAGTCTCTGC; | (SEQ ID NO: 1) |
| | (downstream)-GGTCCAGGATTCTGCCTATGA. | (SEQ ID NO: 2) |
| HCNP: | (upstream)-TGGACGAGCTGGGCAAAGTGC; | (SEQ ID NO: 3) |
| | (downstream)-CCTGCTCGTACACCAGCCAGA. | (SEQ ID NO: 4) |
| NGAL: | (upstream)-CTCAGAACTTGATCCCTGCCC; | (SEQ ID NO: 5) |
| | (downstream)-CCAGCCCTGGAGCTTGGAACA. | (SEQ ID NO: 6) |
| adipsin: | (upstream)-TGCAGAGTGTAGTGCCTCACC; | (SEQ ID NO: 7) |
| | (downstream)-GCAGGTTGTCCGGTTCATGAT. | (SEQ ID NO: 8) |
| haptoglobin: | (upstream)-TGTTGTCACTCTCCT; | (SEQ ID NO: 9) |
| | (downstream)-CCAGCGACTGTGTTCACCCAT. | (SEQ ID NO: 10) |
| Acrp30: | (upstream)-TATCGCTCAGCGTTCAGTGTG; | (SEQ ID NO: 11) |
| | (downstream)-GGCCTGGTCCACATTCTTTTC. | (SEQ ID NO: 12) |
| TBP: | (upstream)-ACCCTTCACCAATGACTCCTATG; | (SEQ ID NO: 13) |
| | (downstream)-ATGATGACTGCAGCAAATCGC. | (SEQ ID NO: 14) |

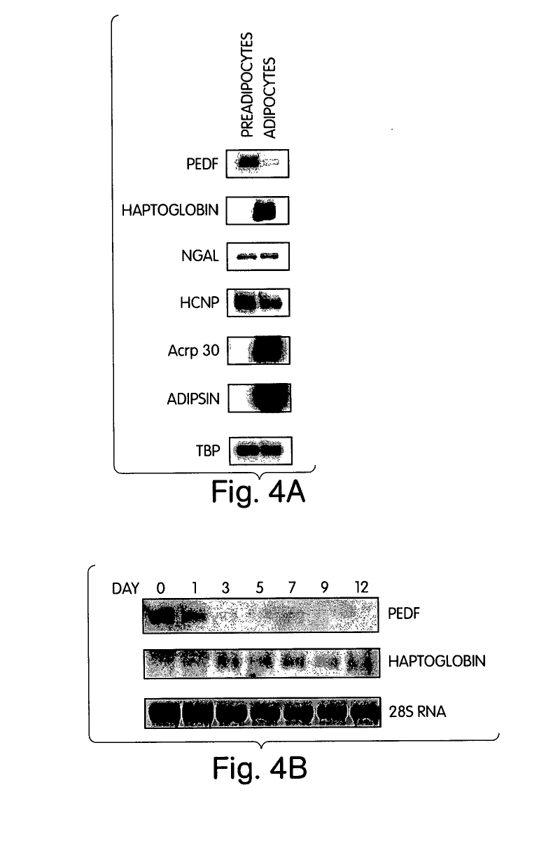

RT-PCR analysis of PEDF revealed that this factor is also differentially expressed at the RNA level. PEDF transcript is detectable in preadipocytes but not in day 9 adipocytes (FIG. 4A). Analysis of haptoglobin RNA expression indicates that this factor is, also differentially expressed at the level of the RNA. Haptoglobin transcript is detectable in day 9 adipocytes but not in preadipocytes (FIG. 4A).

In contrast to these two factors which are differentially expressed at both the RNA and the protein level, the proteomics based approach of the invention also identified two factors which are differentially expressed only at the protein level. RT-PCR analysis of NGAL and HCNP revealed that although these proteins are up-regulated during adipogenesis, there is no change in the expression of RNA during adipogenesis (FIG. 4A). This result suggests that the differential expression of NGAL and HCNP observed during adipogenesis is likely due to post-transcriptional regulation.

Analysis of the RNA expression of these factors confirm that the proteomics based approach described here identified a full range of factors involved in adipogenesis. The invention identified factors expressed preferentially in preadipocytes, factors expressed preferentially in differentiated adipocytes, factors regulated at the transcriptional level, and factors regulated post-transcriptionally.

(b) Northern blot analysis: RNA was isolated from preadipocytes and adipocytes over several days of the differentiation protocol. 20 μg of total RNA was resolved on a denaturing gel containing 1.2% agarose, 20 mM MOPS, pH 7.0, 5 mM Na acetate, 1 mM EDTA, transferred to a Hybond membrane (Amersham Pharmacia) and immobilized by UV cross-linking. Probe fragments corresponding to PEDF and haptoglobin were labeled with Prime-It RmT Random primer labeling kit (Stratagene) using α-³²P dCTP (6000 Ci/mmol) (NEN Life Science Products) and hybridization was performed overnight at 42° C. in a buffer containing 50% deionized formamide, 2.5×Denhardt's solution, 0.38% SDS, 50% dextran sulfate, 2.5×SSPE and 0.1 mg/ml salmon sperm DNA.

Northern blot analysis of PEDF transcript demonstrates that PEDF RNA is abundant in preadipocytes, and is quickly down regulated such that transcripts are barely detectable by day 3 of the differentiation protocol (FIG. 4B). This analysis demonstrates that both PEDF RNA and protein are differentially expressed during adipogenesis, and this time course provides an estimate of when during adipogenesis PEDF is down regulated.

Northern blot analysis of haptoglobin demonstrates that this factor is quickly up-regulated during adipogenesis. By day 3 of the differentiation protocol, the upregulation of haptoglobin transcript is readily detectable (FIG. 4B).

### Example 5

Identification and Quantitation of Secreted Proteins Using LC MS/MS—A Second Proteomics Based Approach

We employed a second proteomics based approach to identify secreted proteins expressed in adipocytes. This approach combines liquid chromatography and mass spectrometry, and avoids the step of separating proteins by gel electrophoresis.

Day 9 adipocytes were cultured as described above. Culture supernatant was subjected to trypsin digestion in solution, loaded onto a nano LC column, sequentially eluted from the column, and fragmented by an on-line mass spectrometer (Ducret et al., 1998; Washburn et al., 2001). Briefly, LC MS/MS analysis was performed on an Agilent Capillary LC system coupled to a quadrupole time-of-flight mass spectrometer (PE Sciex QSTAR Pulsar). The sample was off-line loaded onto a column packed with a 5 um Zorbax C18 resin. Peptides were eluted using a 7%-40% gradient of organic phase over 150 minutes. Buffer A was 0.4% acetic acid, 0.005% HFBA, and buffer B was 90% acetonitrile, 0.4% acetic acid, 0.005% HFBA. The mass spectrometry data was obtained in pulsing mode using Information Dependent Acquisition based on one second MS survey scan followed by up to three MS/MS scans of two seconds each. The data was searched against a non-redundant protein database using MASCOT.

From this analysis, we identified additional secreted factors not previously discovered using the gel electrophoresis based approach described in Example 2. Note that this method identified three factors previously implicated in adipogenesis, as well as several proteins whose role in adipogenesis had not been appreciated (Summarized in Table 2). The identification of both previously recognized and novel factors involved in adipogenesis demonstrates the effectiveness of this gel free method in gaining a more complete understanding of adipogenesis.

It is important to note that the described method was performed only on supernatant from day 9 adipocytes. Therefore, this data provided extensive information concerning proteins secreted by adipocytes, however it did not provide information concerning proteins secreted by preadipocytes or information concerning differential expression between proteins secreted by these two cell populations. The invention also contemplates the application of this approach to the assessment of differential protein expression in different populations of adipocytes in different differentiation stages, or from different sources/origins. Such comparison can be achieved by obtaining the profile of proteins/factors present in a first sample, and compare it with that of a second sample. If difference in expression of a certain protein in two samples is observed, and if that difference exceeds a predetermined/selected threshold value, such as 20%, 30%, 40%, 50%, 60%, 80%, 1-fold, 2-fold, 3-fold, 4-fold, 5-fold, 10-fold, 100-fold, 200-fold, or more increases, etc., that protein is identified as a differentially expressed protein between the two test samples.

For relative quantitative protein analysis of the secreted factors using the SILAC method (Ong et al., 2002), supernatants from non-labeled preadipocytes and labeled adipocytes were collected. The supernatants were mixed in a 1:1 ratio according to protein concentration and subjected to trypsin digestion followed by LC-MS/MS analysis. Results for a selected list of proteins differentially expressed in preadipocytes and mature adipocytes are shown in Table 3.

### Example 6

Novel Factors Identified by LC MS/MS

The approach described in Example 5 identified several proteins previously implicated in adipogenesis, as well as several proteins whose role in adipogenesis had not been previously appreciated. The following factors were identified in adipocytes for the first time using the proteomics based approach of the present invention.

(a) Stromal Cell Derived Factor (SDF-1)

SDF-1 is a CXC chemokine, and a ligand for CXCR4/fusin. The significance of SDF-1 in the immune system has long been appreciated. This factor stimulates proliferation of B-cells, and synergistically augments the ability of IL-7 to stimulate B-cell proliferation (Tashiro et al., 1993; Nagasawa et al., 1994; Oberlin et al., 1996; Bleul et al., 1996). More recently, an anti-infection activity of SDF-1 has been identified. Cells transfected with SDF-1 appear to be resistant to infection by lymphocytic HIV strains (Oberlin et al., 1996; Bleul et al., 1996).

The identification of a factor known for its roles in proliferation and cell survival in the immune system in adipocytes suggests that SDF-1 may help to regulate cell proliferation or behavior is adipose tissue as well.

(b) Calumenin and Calvasculin

Calumenin and calvasculin are two calcium binding proteins expressed in adipocytes (Jackson-Grusby et al., 1987; Yabe et al., 1997). The expression of two calcium binding proteins in adipocytes suggests that utilization and responsiveness to calcium may be an important mechanism of regulation in adipose tissue, and the identification of such proteins offers novel methods to modulate the growth, proliferation, differentiation, or survival of adipocytes.

(c) Colligen-1

Colligen-1 is a protease inhibitor whose role in adipogenesis had not been recognized (Abrahamson et al., 1987; Clarke and Sanwal, 1992). Interestingly, it is not the only protease inhibitor isolated by the invention. Cystatin-C is a second protease inhibitor identified as a factor secreted by adipocytes. Cystatin-C had been previously identified in a cDNA based screen (Tsuruga et al., 2000). Thus, our results confirm that cystatin-C is regulated during adipogenesis at both the RNA and protein levels, and also identify second protease inhibitor expressed during adipogenesis: colligen-1.

(d) Gelsolin

Gelsolin is an actin binding protein found in plasma and other tissues (Kwiatkowski et al., 1986), Given the extensive cellular remodeling that accompanies adipogenesis, it is not surprising that many extracellular matrix related proteins are differentially expressed during adipogenesis. Many such molecules were identified using the approaches describes herein, and manipulation of extra-cellular matrix molecules, with there concomitant effects on cell shape, may present novel means for regulating the growth, proliferation, differentiation, and survival of adipocytes.

(e) Osteoblast Specific Factor 2

Osteoblast specific factor 2 (fasciclin I-like) is a protein highly expressed in bone and lung tissues that functions as an adhesion molecule in bone formation (Takeshita et al., 1993). The human homologue periostin is demonstrated to be secreted and upregulated in epithelial ovarian tumors and to serve as a diagnostic marker for cell lung carcinomas (Sasaki et al., 2001, Gillan et al., 2002). In relation to the adipocyte conversion process osteoblast specific factor 2 has been identified as an upregulated factor that plays a role in extracellular matrix rearrangements and as a regulator of cell migration.

(f) Follistatin-Like Protein

Follistatin-like protein binds and neutralizes both activin, a member of the transforming growth factor-beta and bone morphogenic protein-2, thereby regulating the signal transduction pathways induced by these factors (Tsuchida et al., 2000). Follistatin-like protein has been found to be upregulated in adipocytes and plays an important role in adipogenesis, since TGFbeta and BMP-2 are well known inhibitory factors of adipocyte differentiation.

(g) Calgizzarin

Calgizzarin belongs to the S-100 family and contains two calcium-binding domains. It is a cytokine that activates host immune-response mechanisms by activating endothelial monocytes. In addition, it has been found to be up-regulated in breast and colon cancer (Tanaka et al., 1995). Calgizzarin is downregulated during adipocyte differentiation, suggesting involvment in calcium-mediated signaling.

### REFERENCES

Abrahamson, M., Grubb, A., Olafsson, I., Lundwall, A. (1987). FEBS Lett 216, 229-233.

Aratani, Y., and Kitagawa, Y. (1988). Journal of Biological Chemistry 263, 16163-16169.

Auwerx, J., and Staels, B. (1998). Lancet 351, 737-742.

Baldo, A., Sniderman, A. D., St-Luce, S., Avramoglu, R. K., Maslowska, M., Hoang, B., Monge, J. C., Bell, A., Mulay, S., Cianflone, K. (1993). Journal of Clinical Investigation 92, 1543-1547.

Bleul, C. C., Farzan, M., Choe, H., Parolin, C., Clark-Lewis, I., Sodrowski, J., Springer, T. A. (1996). Nature 382, 829-833.

Bundgaard, J. R., Sengelov, H., Borregaard, N., Kjeldsen, L. (1994). Biochem Biophys Res Commun 202, 1468-1475.

Calle, E., Thun, M., Petrelli, J., Rodriquez, C., Heath, C. (1999). New England Journal of Medicine 341, 1097-1105.

Cao, W., Tombran-Tink, J., Chen, W., Mrazek, D., Elias, R., McGinnis, J. F. (1999). Journal of Neuroscience Research 57, 789-800.

Chader, G. L. (2001). Proceedings of the National Academy of Science 98, 2122-2124.

Choy, L. N., Rosen, B. S., Spiegelman, B. M. (1992). Journal of Biological Chemistry 267, 12736-12741.

Cianflone, K., Roncari, D. A., Maslowska, M., Baldo, A., Forden, J., Sniderman, A. D. (1994). Biochemistry 33, 9489-9495.

Clarke, E. P., and Sanwal, B. D. (1992). Biochim Biophys Acta 1129, 246-248.

Colditz, G. A., Willett, W. C., Rotnitsky, A., Manson, J. E. (1995). Archives of International Medicine 122, 481-486.

Cook, K. S., Min, H. Y., Johnson, D., Chaplinsky, R. J., Flier, J. S., Hunt, C. R., Spiegelman, B. M. (1987). Science 237, 402-205.

Dagogo-Jack, S. (2001). Current Drug Targets 2, 181-195.

Dawson, D. W., Volpert, O. V., Gillis, P., Crawford, S. E., Xu, H., Benedict, W., Bouck, N. P. (1999). Science 285, 245-248.

De Divitiis, O. (1981). Circulation 64, 477-482.

Dietz, W. H. (1994). American Journal of Clinical Nutrition 59, 829-840.

Dilley, A. V., Patel, D. L., Hicks, M. J., Brandt, M. L. (2001). Journal of Pediatric Surgery 36, 229-231.

Ducret, A., Van Oostveen, I., Eng, J. K., Yates, J. R., 3rd, Aebersold, R. (1998). Protein Science 7, 706-719.

Flower, D. R., North, A. C., Attwood, T. K. (1991). Biochem Biophys Res Commun 180, 69-74.

Forrester, T. et al. in The Origins and Consequences of Obesity (eds Chadwick, D. J. & Cardeau, G.) 17-31.

Friedman, J. M. (2000). Nature 404, 632-634.

Friedman, J. M., Halaas, J. (1998). Nature 395, 763-770.

Friedrichs, W. E., Navarijo-Ashbaugh, A. L., Bowman, B. H., Yang, F. (1995). Biochem Biophys Res Commun 209, 250-256.

Fruebis, J., Tsao, T. S., Javorschi, S., Ebbets-Reed, D., Erickson, M. R., Yen, F. T., Bihain, B. E., Lodish, H. F. (2001). Proceedings of the National Academy of Science 98, 2005-2010.

Gillan, L., Matei, D., Fishman, D. A., Gerbin, C. S., Karlan, B. Y., Chang, D. D. (2002). Cancer Research 62, 5358-5364.

Gobom, J., Nordhoff, E., Mirgorodskaya, E., Ekman, R., Roepstorff, P. (1999). Journal of Mass Spectrometry 34, 105-116.

Grunstein, R. R. in Clinical Obesity (eds Kopelman and Stock) 248-289 (Blackwell Science, Oxford, 1998).

Halaas, J. L., et al. (1995). Science 269, 543-546.

Halaas, J. L., et al. (1997). Proceedings of the National Academy of Science 94, 8878-8883.

Hanley, J. M., Haugen, T. H., and Heath, E. C. (1983). Journal of Biological Chemistry 258, 7858-7869.

Hansen, J. B., Petersen, R. K., Larsen, B. M., Bartkova, J., Alsner, J., Kristiansen, K. K. (1999) Journal of Biological Chemistry 274, 2386-2393.

Hodge A. M. (1995). Obesity Research 3 (Supplement 2) 77s-87s.

Houenou, L. J., D'Costa, A. P., Li, L., Turgeon, V. L., Enyadike, C., Alberdi, E., Becerra, S. P. (1999). Journal of Comparative Neurology 412, 506-514.

Hraba-Renevey, S., Turler, H., Kress, M., Salomon, C., Weil, R. (1989). Oncogene 4, 601-608.

Hu, E., Liang, P., Spiegelman, B. M. (1996). Journal of Biological Chemistry 271, 10697-10703.

Hubert, H. B. (1986). Annual Review of Public Health 7, 493-502.

Hubert., H. B., Feinleib, M., McNamara, P. M. Castelli, W. P. (1983). Circulation 67, 968-977.

Jackson-Grusby, L. L., Swiergiel, J., Linzer, D. I. (1987). Nucleic Acids Research 15, 6677-6690.

Kitagawa, K., Rosen, B. S., Spiegelman, B. M., Lienhard, G. E., Tanner, L. I. (1989). Biochim Biophys Acta 1014, 83-89.

Kjeldsen, L., Cowland, J. B., Borregaard, N. (2000). Biochim Biophys Acta 1482, 272-283.

Kolterman, O. G., Insel, J., Sackow, M., Olefsky, M. (1980). Journal of Clinical Investigation 65, 1272-1284.

Kopelman, P. G. (2000). Nature 404, 635-643.

Kopelman, P. G. (1992). International Journal of Obesity 16 (Supplement 2) S37-S42.

Kotani, K., et al. (1997). International Journal of Obesity 21, 912-921.

Kwiatkowski, D. J., Stossel, T. P., Orkin, S. H., Mole, J. E., Colten, H. R., Yin, H. L. (1986). Nature 323, 455-458.

Lakshmiah, S. R., Scott, K. W., Whear, N. M., Monoghan, A. (2000). International Journal of Oral and Maxillofacial Surgery 29, 445-446.

Lawrence I. C., Fiocchi, C., Chakravarti, S. (2001). Human Molecular Genetics 10, 445-456.

Lew, E. A. (1985). Annals of Internal Medicine 103, 1024-1029.

Lewandowski, P. J., Weiner, S. D. (1996). Clinical Orthopaedics 330, 198-201.

Licata, G. (1991). International Journal of Obesity 15, 295-302.

Marinkovic, S., Baumann, H. (1990). Molecular Cellular Biology 10, 1573-1583.

Maslowska, M., Sniderman, A. D., Germinario, R., Cianflone, K. (1997). Int J Obes Relat Metab Disord 21, 261-266.

Murray, I., Parker, R. A., Kirchgessner, T. G., Tran, J., Zhang, Z. J., Westerlund, J., Cianflone, K. (1997). Journal of Lipid Research 38, 2492-2501.

Musaiger, A. in Diet-Related Non-communicable Disease in the Arab Countries of the Gulf (eds Musaiger and Miladi) 99-117 (Food and Agriculture Organization of the United Nations, Cairo, 1996).

Nagasawa, T., Kikutani, H., and Kishimoto, T. (1994). Proceedings of the National Academy of Science 91, 2305-2309.

Oberlin, E., Amara, A., Bachelerie, F., Bessia, C., Virelizier, J. L., Arenzana-Seisdedos, F., Schwartz, O., Heard, J. M., Clark-Lewis, I., Legler, D. F., Loetscher, M., Baggiolini, M., Moser, B. (1996). Nature 382, 833-835.

Ogata, N., Wang, L., Jo, N., Tombran-Tink, J., Takahashi, K., Mrazek, D., Matsumura, M. (2001). Current Eye Research 22, 245-252.

Ojika, K., Kojima, S., Ueki, Y., Fukushima, N., Hayashi, K., Yamamoto, M. (1992). Brain Res 572, 164-171.

Ojika, K., Mitake, S., Kamiya, T., Kosuge, N., Taiji, M. (1994). Brain Res Dev Brain Res 79, 1-9.

Ojika, K., Tsugu, Y., Mitake, S., Otsuka, Y., Katada, E. (1998). Brain Res Dev Brain Res 106, 173-180.

Oliveira, A. M., Nascimento, A. G., Lloyd, R. V. (2001). Mod Pathology 14, 549-555.

Ong, S E., Blagoev, B., Kratchmarova, I., Kristensen, D. B., Steen, H., Pandey, A., Mann, M. (2002). Mol Cell Proteomics. 5, 376-386.

Popkin, B. M. (1994). Nutrition Review 52, 285-298.

Rosen, E. D., Sarraf, P., Troy, A. E., Bradwin, G., Moore, K., Milstone, D. S., Spiegelman, B. M., Mortensen, R. M. (1999). Molecular Cell 4, 611-617.

Rosen, E. D., Walkey, C. J., Puigserver, P., Spiegelman, B. M. (2000). Genes and Development 14, 1293-1307.

Sasaki, H., Auclair, D., Fukai, I., Kirijama, M., Yamakawa, Y., Fujii, Y., Chen, L. B. (2001) Cancer 92, 843-848

Scherer, P. E., Bickel, P. E., Kotler, M., Lodish, H. F. (1998). Nature Biotechnology 16, 581-586.

Scherer, P. E., Williams, S., Fogliano, M., Baldini, G., Lodish, H. F. (1995). Journal of Biological Chemistry 270, 26746-26749.

Shirozu, M., Tada, H., Tashiro, K., Nakamura, T., Lopez, N. D., Nazarea, M., Hamada, T., Sato, T., Nakano, T., and Honjo, T. (1996). Genomics 37, 273-280.

Seidell, J. C. (1996). International Journal of Obesity 19(Supplement), S13-S16.

Selvarajan, S., Lund, L. R., Takeuchi, T., Craik, C. S., Werb, Z. (2001). Nature Cell Biology 3, 267-275.

Shevchenko, A., Wilm, M., Vorm, O., Mann, M. (1996). Analytic Chemistry 68, 850-858.

Society of Actuaries. Build Study of 1979 (Recording and Statistical Corporation, 1980).

Spiegelman, B. M., Frank, M., Green, H. (1983). Journal of Biological Chemistry 258, 10083-10089.

Steele, F. R., Chader, G. J., Johnson, L. V., Tombran-Tink, J. (1993). Proceedings of the National Academy of Science 90, 1526-1530.

Stellmach, V. V., Crawford, S. E., Zhou, W., Bouck, N. (2001). Proceedings of the National Academy of Science 98, 2593-2597

Taiji, M., Tohdoh, N., Ojika, K. (1996). Journal of Neuroscience Research 45, 202-215.

Takeshita, S., Kikuno, R., Tezuka, K., Amann, E. (1993) Biochem. Journal 294, 271-278.

Tanaka, M., Adzuma, K., Iwami, M., Yoshimoto, K., Monden, Y., Itakura, M. (1995) Cancer Letters 89, 195-200

Tashiro, K., Tada, H., Heilker, R., Shirozu, M., Nakano, T., Honjo, T. (1993). Science 261, 600-603.

Tombran-Tink, J., Chader, G. G., and Johnson, L. V. (1991). Experimental Eye Res 53, 411-414.

Troiano, R., Flegal, K. (1998). Pediatrics 101, 497-504.

Tsuchida, K., Arai, K Y., Kuramoto, Y., Yamakawa, N., Hasegawa, Y., Sugino, H. (2000) Journal of Biological Chemistry 275, 40788-40796.

Tsugu, Y., Ojika, K., Matsukawa, N., Iwase, T., Otsuka, Y., Katada, E., Mitake, S. (1998). European Journal of Neurology 5, 561-569.

Tsuruga, H., Kumagai, H., Kojima, T., Kitamura, T. (2000). Biochem Biophys Res Commun 272, 293-197.

Washburn, M. P., Wolters, D., Yates, J. R., 3rd. (2001). Nature Biotechnology 19, 242-247.

Weiner, F. R., Shah, A., Smith, P. J., Rubin, C. S., Zern, M. A. (1989). Biochemistry 28, 4094-4099.

Willet, W. C. (1995). Journal of the American Medical Association 273, 461-465.

Wilm, M., Shevchenko, A., Houthaeve, T., Breit, S., Schweigerer, L., Fotsis, T., Mann, M. (1996). Nature 379, 466-469.

Yabe, D., Nakamura, T., Kanazawa, N., Tashiro, K., Honjo, T. (1997). Journal of Biological Chemistry 272, 18232-18239.

Yoshikawa, H., Myoui, A., Ochi, T., Araki, N., Ueda, T., Kudawara, I., Nakanishi, K., Tanaka, H., Nakamura, H. (1999). Seminars in Musculoskeletal Radiology 3, 183-190.

Yu, S. J., Boudreau, F., Desilets, A., Houde, M., Rivard, N., Asselin, C. (1999). Biochem Biophys Res Commun 259, 544-549.

Zhou, L., Halvorsen, Y. -D., Cryan, E. V., Pelton, P. D., Burris, T. P., Demarest, K. T. (1999). Biotechnology Techniques 13, 513-517.

All of the patents, applications, and publications cited above are hereby incorporated by reference herein.

TABLE 1

| Downregulated secreted proteins | Band number | Database accession # |
|---|---|---|
| Fibronectin | 1 | P11276 |
| Pigment epithelium derived | 8 | AAC05731 |
| factor | | |
| Type I collagen alpha 2 | 9 | NP_031769 |
| Upregulated secreted proteins | Band number | Database accession # |
| Type VI collagen alpha 3 | 2, 3, 4 | AAC23667 |
| Complement factor C3 | 5, 7, 10, 11 | AAC42013 |
| Entactin/Nidogen | 6 | NP_035047 |
| Haptoglobin | 12, 13, 14 | NP_059066 |
| Adipsin | 12, 14 | NP_038487 |
| Adipocyte complement-related | 15 | AAA80543 |
| protein (ACRP 30) | | |
| Hippocampal cholinergic | 16 | BAB03276 |
| neurostimulating peptide | | |
| precursor protein (HCNP) | | |
| Neutrophil gelatinase associated | 16 | CAA32762 |
| lipocalin precursor (NGAL) | | |

TABLE 2

| Protein identified | Database accession # |
|---|---|
| Resistin | NP_075360 |
| SPARC/osteonectin | NP_033268 |
| Cystatin 3 | NP_034106 |
| SDF-1 | P40224 |
| Calumenin | NP_031620 |
| Gelsolin | NP_034484 |
| Colligin-1 | NP_004344 |
| Matrix-metalloproteinase 2 (MMP-2) | NP_032636 |
| Mouse placental Calcium binding | P07091 |
| protein | |
| Type III Collagen alpha 1 | P08121 |
| Type VI Collagen alpha 1 | NP_034063 |
| Type VI Collagen alpha 2 | S21369 |

TABLE 3

| | | Protein ratio |
|---|---|---|
| | No. of peptides | (adipocytes:pre- |
| Protein name | used for quantitation | adipocytes) |
| Collagen type VI alpha 3 | 15 | 1.68 |
| Fibronectin | 10 | 0.32 |
| Laminin beta-1 chain | 4 | 2.72 |
| Laminin alpha-4 chain | 6 | 2.74 |
| Collagen type XII alpha 1 | 4 | 0.33 |
| Biglycan | 9 | 0.90 |
| Laminin gamma-1 chain | 1 | 1.29 |
| Complement factor C3 | 15 | >10 |
| Osteoblast specific factor 2 | 9 | 2.85 |
| fasciclin I-like) | | |
| Matrix metalloproteinase 2 | 4 | 0.75 |
| Nidogen/entactin | 1 | 1.96 |
| Collagen type VI alpha 1 | 7 | 1.97 |
| Collagen type I alpha 2 | 3 | 5.47 |
| Thrombospondin 2 | 3 | 1.87 |
| Complement component 4 | 1 | 2.71 |
| Collagen binding protein 1 | 8 | 1.63 |
| SPARC | 7 | 2.2 |
| Lipoprotein lipase | 5 | >10 |
| Angiotensinogen | 5 | >10 |
| PEDF/SDF-3 | 6 | 0.81 |
| ACRP 30 (Adiponectin) | 4 | >10 |
| Serine protease inhibitor 3 | 2 | 0.61 |
| Procollagen C-proteinase | 3 | 0.96 |
| enhancer protein | | |
| Cathepsin D | 2 | 3.24 |
| Alpha-1-acid glycoprotein | 1 | >10 |
| Cyclophilin A | 5 | 0.42 |
| Cofilin 1, non-muscle | 8 | 0.16 |
| Cyclophilin B | 1 | 1.06 |
| Cyclophilin C | 1 | 1.48 |
| Adipsin | 1 | >10 |
| Phospholipase A2 | 1 | 1.66 |
| Follistatin-like protein | 1 | 1.71 |
| Platelet-activating factor | 1 | 0.83 |
| acetylhydrolase alpha 2 | | |
| Sulfated glycoprotein | 4 | 2.52 |
| Similar to Pigment epithelium- | 2 | 1.02 |
| derived factor | | |
| Placental calcium-binding | 2 | 0.19 |
| protein | | |
| Cystatin C | 3 | >10 |
| Calgizzarin | 6 | 0.5 |

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for identifying a protein differentially expressed between a first and a second populations of cells of adipose lineage, comprising:
(i) obtaining a first protein sample from said first populations of cells, and a second protein sample from said second populations of cells;
(ii) separating proteins in said first and second protein samples;
(iii) identifying and isolating one or more proteins, if any, differentially expressed in said first and said second populations of cells; and
(iv) determining, by mass spectrometry, the identity/sequence of said differentially expressed proteins isolated in (iii).

2. The method of claim 1, wherein protein samples of step (i) comprise secreted proteins.

3. The method of claim 1, wherein said first and said second populations of cells are each independently of embryonic, post-natal, or adult origin.

4. The method of claim 1, wherein both said first and said second populations of cells are derived from mammalian species.

5. The method of claim 4, wherein said mammalian species is non-human primate.

6. The method of claim 4, wherein said mammalian species is human.

7. The method of claim 1, wherein step (ii) is effectuated by SDS-PAGE.

8. The method of claim 1, wherein step (ii) is effectuated by nono-Liquid Chromatography coupled directly to mass spectrometer (nLC-MS).

9. The method of claim 1, wherein said first and second protein samples obtained in step (i) are digested before separation in step (ii).

10. The method of claim 7, wherein proteins identified and isolated in step (iii) is digested by in-gel digestion.

11. The method of claim 1, wherein in step (iii), proteins are identified as differentially expressed based on quantitation or semi-quantitation of separated proteins.

12. The method of claim 11, wherein said quantitation or semi-quantitation is carried out by visual comparison.

13. The method of claim 1, wherein step (iv) is effected by tandem mass spectrometry (MS/MS).

14. A method for identifying an agent capable of modulating adipogenesis, comprising:
(i) identifying, using the method of claim 1, one or more proteins differentially expressed between pre-adipocytes and adipocytes;
(ii) contacting cells in culture with said protein(s), wherein said cells are: preadipocytes, adipocytes, fibroblasts, embryonic stem cells, or adult stem cells; and,
(iii) analyzing the cells in culture for changes in proliferation, differentiation, survival, or expression of adipogenesis marker genes,
wherein a change in proliferation, differentiation, survival, or expression of adipogenesis marker genes after contacting said cells with said protein(s) indicates that said protein(s) is an agent capable of modulating adipogenesis.

15. The method of claim 14, wherein said agent is an adipogenic agonist which increases or potentiates the growth, proliferation, differentiation, or survival of said cells.

16. The method of claim 14, wherein said agent is an adipogenic antagonist which decreases or inhibits the growth, proliferation, differentiation, or survival of said cells.

17. The method of claim 14, wherein said one or more proteins differentially expressed between pre-adipocytes and adipocytes is selected from: pigment epithelium derived factor (PEDF), haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

18. The method of claim 14, further comprising formulating said agent with a pharmaceutically acceptable carrier or excipient.

19. A method for increasing adipogenesis in a cell, comprising contacting said cell with an effective amount of an adipogenic agonist of claim 15.

20. A method for decreasing adipogenesis in a cell, comprising contacting said cell with an effective amount of an adipogenic antagonist of claim 16.

21. A method for treating a patient having a condition characterized by hyper-adipogenesis, comprising treating the patient with an effective amount of an adipogenic agonist of claim 15.

22. A method for treating a patient having a condition exacerbated by obesity or excess body fat, or a condition characterized by hypo-adipogenesis, comprising treating the patient with an effective amount of an adipogenic antagonist of claim 16.

23. The method of claim 21, wherein the condition is obesity, hyper-lipidemia, hyper-cholesterolemia, hypertriglyceridemia, liposarcoma, lipoma, hibernoma, or lipoblastoma.

24. The method of claim 22, wherein the condition is type II diabetes, high blood pressure, osteoarthritis, asthma, respiratory insufficiency, coronary heart disease, cancer, or sleep apnea.

25. The method of claim 21 or 22, wherein the patient is an animal.

26. The method of claim 25, wherein the animal is a farm animal selected from: cow, pig, sheep, chicken, duck, goat, deer, or buffalo.

27. The method of claim 21 or 22, wherein the patient is a human patient.

28. The method of claim 27, wherein the condition is selected from: malnutrition, anorexia nervosa, bulimia nervosa, low birth weight, wasting associated with AIDS, cancer, or side effects of cancer therapy.

29. The method of claim 27, wherein the patient is a fetus and the adipogenic agonist is administered in utero.

30. A method of modulating adipogenesis in a cell, comprising contacting the cell with an effective amount of an agent selected from: pigment epithelium derived factor (PEDF), haptoglobin, neutrophil gelatinase associated lipocalin, hippocampal cholinergic neurostimulating peptide, stromal cell derived factor-1/pre-B cell growth stimulating factor, calumenin, calvasculin, colligen-1, gelsolin, osteoblast specific factor 2, follistatin-like protein or calgizzarin.

31. A method of determinig the differentiation stage of adipogenesis in a cell, comprising identifying one or more agents whose expression level is substantially changed during adipogenesis, and determining the expression level of said one or more agents during adipogenesis of said cell, thereby determining the differentiation stage of adipogenesis in said cell.

32. A method for quantitating a protein differentially expressed between a first and a second populations of cells of adipose lineage, comprising:
(i) obtaining a first protein sample from said first populations of cells, and a second protein sample from said second populations of cells;
(ii) separating proteins in said first and second protein samples;
(iii) identifying and isolating one or more proteins, if any, differentially expressed in said first and said second populations of cells; and
(iv) determining, by mass spectrometry, the identity and relative quantity of said differentially expressed proteins isolated in (iii).

33. A method of conducting a drug discovery business comprising:
(i) identifying, using the method of claim x, one or more agents capable of modulating adipogenesis;
(ii) conducting therapeutic profiling of agents identified in step (i), or further analogs thereof, for efficacy and toxicity in animals; and
(iii) formulating a pharmaceutical preparation including one or more agents identified in step (ii) as having an acceptable therapeutic profile.

34. The method of claim 33, further comprising a step of establishing a distribution system for distributing the pharmaceutical preparation for sale.

35. The method of claim 33, further comprising a step of establishing a sales group for marketing the pharmaceutical preparation.

36. A method of conducting a target discovery business comprising:
(i) identifying, using the method of claim x, one or more agents capable of modulating adipogenesis;
(ii) (optionally) conducting therapeutic profiling of agents identified in step (i) for efficacy and toxicity in animals; and
(iii) licensing, to a third party, the rights for further drug development and/or sales for gents identified in step (i), or analogs thereof.
